SEQUENCE LISTING (56 sequences)

SEQ ID NO: 1 - protein, 55 aa, Artificial Sequence
  Synthetic peptide sequence
VTKNHAKKDVVVGGKTNTTTSRCTGSNDKRCKHSMVDRGWGNGCGGKGGVTCAMT

SEQ ID NO: 2 - DNA, 1485 bp, Dengue virus type 2
atgcgttgcataggaatatcaaatagagactttgtagaaggggtttcaggaggaagctgggttgacatagtcttagaacatggaagttgtgtgacgacgatggcaaaaaataaaccaacattggattttgaactgataaaaacagaagccaaacaacctgccactctaaggaagtactgtatagaagcaaagctgaccaatacaacaacagaatctcgttgcccaacacaaggggaacccagtctaaatgaagagcaggacaaaaggttcctctgcaaacactccatggtagacagaggatggggaaatggatgtggattatttggaaagggaggcattgtgacctgtgctatgtttacatgcaaaaagaacatggaaggaaaagtcgtgctgccagaaaatttggaatacaccatcgtgataacacctcactcaggagaagagcacgctgtaggtaatgacacaggaaaacatggcaaggaaatcaaaataacaccacagagttccatcacagaagcagaactgacaggctatggcactgtcacgatggagtgctctccgagaacgggcctcgacttcaatgagatggtgctgctgcagatggaagacaaagcttggctggtgcacaggcaatggttcctagacctgccgttaccatggctacccggagcggacacacaaggatcaaattggatacagaaagagacattggtcactttcaaaaatccccacgcgaagaaacaagatgtcgttgttttaggatctcaagaaggggccatgcacacggcactcacaggggccacagaaatccagatgtcatcaggaaacttactgttcacaggacatctcaagtgcaggctgagaatggacaaactacagctcaaaggaatgtcatactctatgtgtacaggaaagtttaaaattgtgaaggaaatagcagaaacacaacatggaacaatagttatcagagtacaatatgaaggggacggctctccatgtaagatcccttttgagataatggatttggaaaaaagacacgtcttaggtcgcctgattacagttaacccgatcgtaacagaaaaagatagcccagtcaacatagaagcagaacctccattcggagacagctacatcatcataggagtagagccgggacaattgaaactcaactggtttaagaaaggaagttccatcggccaaatgtttgagacaacaatgagaggagcgaagagaatggccattttaggtgacacagcctgggattttggatccctgggaggagtgtttacatctataggaaaggctctccaccaagttttcggagcaatctatggggctgcttttagtggggtctcatggactatgaaaatcctcataggagtcatcatcacatggataggaatgaattcacgtagcacctcactgtctgtgtcactagtattggtgggagtcgtgacactgtacctgggagctatggtgcaggct

SEQ ID NO: 3 - protein, 495 aa, Dengue virus type 2
MRCIGISNRDFVEGVSGGSWVDIVLEHGSCVTTMAKNKPTLDFELIKTEAKQPATLRKYCIEAKLTNTTTESRCPTQGEPSLNEEQDKRFLCKHSMVDRGWGNGCGLFGKGGIVTCAMFTCKKNMEGKVVLPENLEYTIVITPHSGEEHAVGNDTGKHGKEIKITPQSSITEAELTGYGTVTMECSPRTGLDFNEMVLLQMEDKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKIVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMFETTMRGAKRMAILGDTAWDFGSLGGVFTSIGKALHQVFGAIYGAAFSGVSWTMKILIGVIITWIGMNSRSTSLSVSLVLVGVVTLYLGAMVQA

SEQ ID NO: 4 - DNA, 242 bp, Artificial Sequence
  Synthetic nucleotide sequence
ccatggcattggtcactttcaaaaatccccacgcgaagaaacaagatgtcgttgttggaggtggaaagctgaccaatacaacaacagaatctcgttgcccaacacaaggggaacccagtctaaatgaagagcaggacaaaaggttcctctgcaaacactctatggtagacagaggatggggaaatggatgtggattatttggaaagggaggcattgtgacctgtgctatgtttacactcgag

SEQ ID NO: 5 - DNA, 7 bp, Artificial Sequence
  Synthetic nucleotide sequence
catggca

SEQ ID NO: 6 - DNA, 48 bp, Dengue virus type 2
ttggtcactttcaaaaatccccacgcgaagaaacaagatgtcgttgtt

SEQ ID NO: 7 - protein, 16 aa, Dengue virus type 2
LVTFKNPHAKKQDVVV

SEQ ID NO: 8 - DNA, 9 bp, Artificial Sequence
  Synthetic nucleotide sequence
ggaggtgga

SEQ ID NO: 9 - protein, 57 aa, Dengue virus type 2
KLTNTTTESRCPTQGEPSLNEEQDKRFLCKHSMVDRGWGNGCGLFGKGGIVTCAMFT

SEQ ID NO: 10 - DNA, 171 bp, Dengue virus type 2
aagctgaccaatacaacaacagaatctcgttgcccaacacaaggggaacccagtctaaatgaagagcaggacaaaaggttcctctgcaaacactccatggtagacagaggatggggaaatggatgtggattatttggaaagggaggcattgtgacctgtgctatgtttaca

SEQ ID NO: 11 - DNA, 6 bp, Artificial Sequence
  Synthetic nucleotide sequence
ctcgag

SEQ ID NO: 12 - DNA, 5664 bp, Artificial Sequence
  Synthetic nucleotide sequence
taatacgactcactataggggaattgtgagcggataacaattcccctctagaaataattttgtttaactttaagaaggagatatacatatgaaatacctgctgccgaccgctgctgctggtctgctgctcctcgctgcccagccggcgatggccatggcattggtcactttcaaaaatccccacgcgaagaaacaagatgtcgttgttggaggtaagctgaccaatacaacaacagaatctcgttgcccaacacaaggggaacccagtctaaatgaagagcaggacaaaaggttcctctgcaaacactctatggtagacagaggatggggaaatggatgtggattatttggaaagggaggcattgtgacctgtgctatgtttacactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaat

SEQ ID NO: 13 - protein, 108 aa, Artificial Sequence
  Synthetic peptide sequence
MKYLLPTAAAGLLLLAAQPAMAMALVTFKNPHAKKQDVVVGGGKLTNTTTESRCPTQGEPSLNEEQDKRFLCKHSMVDRGWGNGCGLFGKGGIVTCAMFTLEHHHHHH

SEQ ID NO: 14 - protein, 83 aa, Artificial Sequence
  Synthetic peptide sequence
LVTFKNPHAKKQDVVVGGKLTNTTTESRCPTQGEPSLNEEQDKRFLCKHSMVDRGWGNGCGLFGKGGIVTCAMFTLEHHHHHH

SEQ ID NO: 15 - DNA, 734 bp, Artificial Sequence
  Synthetic nucleotide sequence
ccatggcaaacattgtgatgacccaatctcccaaatccatgtccatgtcagtaggagagagggtcaccttgacctgcaaggccagtgagaatgtggttacttatgtttcctggtatcaacagaaaccagagcagtctcctaaactgctgatatacggggcatccaaccggtacactggggtccccgatcgcttcacaggcagtggatctgcaacagatttcactctgaccatcagcagtgtgcaggctgaagaccttgcagattatcactgtggacagggttacagctatccgtacacgttcggaggggggaccaagctggaaataaaagagggtaaatcctcaggatcaggctccgaatccaaagtcgacgaggtccagctgcaacaatctggacctgagctggtgaagcctgggacttcagtgaagatatcctgcaagacttctggatacacattcactgaatataccatacactgggtgaagcagagccacggaaagagccttgcgtggattggaggtattgatcctaacagtggtggtactaactacagcccgaacttcaagggcaaggccacattgactgttgacaagtcctccagcacagcctacatggacctccgcagcctgtcatctgaggattctgcagtctacttctgtgcaaggatctatcattacgacggatacttcgatgtctggggcgcagggaccgccgtcaccgtctcctcactcgag

SEQ ID NO: 16 - DNA, 905 bp, Artificial Sequence
  Synthetic nucleotide sequence
ccatggcaaacattgtgatgacccaatctcccaaatccatgtccatgtcagtaggagagagggtcaccttgacctgcaaggccagtgagaatgtggttacttatgtttcctggtatcaacagaaaccagagcagtctcctaaactgctgatatacggggcatccaaccggtacactggggtccccgatcgcttcacaggcagtggatctgcaacagatttcactctgaccatcagcagtgtgcaggctgaagaccttgcagattatcactgtggacagggttacagctatccgtacacgttcggaggggggaccaagctggaaataaaagagggtaaatcctcaggatcaggctccgaatccaaagtcgacgaggtccagctgcaacaatctggacctgagctggtgaagcctgggacttcagtgaagatatcctgcaagacttctggatacacattcactgaatataccatacactgggtgaagcagagccacggaaagagccttgcgtggattggaggtattgatcctaacagtggtggtactaactacagcccgaacttcaagggcaaggccacattgactgttgacaagtcctccagcacagcctacatggacctccgcagcctgtcatctgaggattctgcagtctacttctgtgcaaggatctatcattacgacggatacttcgatgtctggggcgcagggaccgccgtcaccgtctcctcaggtggtggttccggtggtggttccggtggtggttccggtggtggtgaagacccgtgcgcttgcgaatccctggttaaattccaggctaaagttgaaggtctgctgcaggctctgacccgtaaactggaagctgtttccaaacgtctggctatcctggaaaacaccgttgttctcgag

SEQ ID NO: 17 - DNA, 1439 bp, Artificial Sequence
  Synthetic nucleotide sequence
ccatggcaaacattgtgatgacccaatctcccaaatccatgtccatgtcagtaggagagagggtcaccttgacctgcaaggccagtgagaatgtggttacttatgtttcctggtatcaacagaaaccagagcagtctcctaaactgctgatatacggggcatccaaccggtacactggggtccccgatcgcttcacaggcagtggatctgcaacagatttcactctgaccatcagcagtgtgcaggctgaagaccttgcagattatcactgtggacagggttacagctatccgtacacgttcggaggggggaccaagctggaaataaaagagggtaaatcctcaggatcaggctccgaatccaaagtcgacgaggtccagctgcaacaatctggacctgagctggtgaagcctgggacttcagtgaagatatcctgcaagacttctggatacacattcactgaatataccatacactgggtgaagcagagccacggaaagagccttgcgtggattggaggtattgatcctaacagtggtggtactaactacagcccgaacttcaagggcaaggccacattgactgttgacaagtcctccagcacagcctacatggacctccgcagcctgtcatctgaggattctgcagtctacttctgtgcaaggatctatcattacgacggatacttcgatgtctggggcgcagggaccgccgtcaccgtctcctcaggcggtggcgagcccaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaactcgag

SEQ ID NO: 18 - DNA, 6159 bp, Artificial Sequence
  Synthetic nucleotide sequence
ccatggcaaacattgtgatgacccaatctcccaaatccatgtccatgtcagtaggagagagggtcaccttgacctgcaaggccagtgagaatgtggttacttatgtttcctggtatcaacagaaaccagagcagtctcctaaactgctgatatacggggcatccaaccggtacactggggtccccgatcgcttcacaggcagtggatctgcaacagatttcactctgaccatcagcagtgtgcaggctgaagaccttgcagattatcactgtggacagggttacagctatccgtacacgttcggaggggggaccaagctggaaataaaagagggtaaatcctcaggatcaggctccgaatccaaagtcgacgaggtccagctgcaacaatctggacctgagctggtgaagcctgggacttcagtgaagatatcctgcaagacttctggatacacattcactgaatataccatacactgggtgaagcagagccacggaaagagccttgcgtggattggaggtattgatcctaacagtggtggtactaactacagcccgaacttcaagggcaaggccacattgactgttgacaagtcctccagcacagcctacatggacctccgcagcctgtcatctgaggattctgcagtctacttctgtgcaaggatctatcattacgacggatacttcgatgtctggggcgcagggaccgccgtcaccgtctcctcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgaaatacctgctgccgaccgctgctgctggtctgctgctcctcgctgcccagccggcgatgg

SEQ ID NO: 19 - DNA, 6330 bp, Artificial Sequence
  Synthetic nucleotide sequence
ccatggcaaacattgtgatgacccaatctcccaaatccatgtccatgtcagtaggagagagggtcaccttgacctgcaaggccagtgagaatgtggttacttatgtttcctggtatcaacagaaaccagagcagtctcctaaactgctgatatacggggcatccaaccggtacactggggtccccgatcgcttcacaggcagtggatctgcaacagatttcactctgaccatcagcagtgtgcaggctgaagaccttgcagattatcactgtggacagggttacagctatccgtacacgttcggaggggggaccaagctggaaataaaagagggtaaatcctcaggatcaggctccgaatccaaagtcgacgaggtccagctgcaacaatctggacctgagctggtgaagcctgggacttcagtgaagatatcctgcaagacttctggatacacattcactgaatataccatacactgggtgaagcagagccacggaaagagccttgcgtggattggaggtattgatcctaacagtggtggtactaactacagcccgaacttcaagggcaaggccacattgactgttgacaagtcctccagcacagcctacatggacctccgcagcctgtcatctgaggattctgcagtctacttctgtgcaaggatctatcattacgacggatacttcgatgtctggggcgcagggaccgccgtcaccgtctcctcaggtggtggttccggtggtggttccggtggtggttccggtggtggtgaagacccgtgcgcttgcgaatccctggttaaattccaggctaaagttgaaggtctgctgcaggctctgacccgtaaactggaagctgtttccaaacgtctggctatcctggaaaacaccgttgttctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgaaatacctgctgccgaccgctgctgctggtctgctgctcctcgctgcccagccggcgatgg

SEQ ID NO: 20 - DNA, 6864 bp, Artificial Sequence
  Synthetic nucleotide sequence
catggcaaacattgtgatgacccaatctcccaaatccatgtccatgtcagtaggagagagggtcaccttgacctgcaaggccagtgagaatgtggttacttatgtttcctggtatcaacagaaaccagagcagtctcctaaactgctgatatacggggcatccaaccggtacactggggtccccgatcgcttcacaggcagtggatctgcaacagatttcactctgaccatcagcagtgtgcaggctgaagaccttgcagattatcactgtggacagggttacagctatccgtacacgttcggaggggggaccaagctggaaataaaagagggtaaatcctcaggatcaggctccgaatccaaagtcgacgaggtccagctgcaacaatctggacctgagctggtgaagcctgggacttcagtgaagatatcctgcaagacttctggatacacattcactgaatataccatacactgggtgaagcagagccacggaaagagccttgcgtggattggaggtattgatcctaacagtggtggtactaactacagcccgaacttcaagggcaaggccacattgactgttgacaagtcctccagcacagcctacatggacctccgcagcctgtcatctgaggattctgcagtctacttctgtgcaaggatctatcattacgacggatacttcgatgtctggggcgcagggaccgccgtcaccgtctcctcaggcggtggcgagcccaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgaaatacctgctgccgaccgctgctgctggtctgctgctcctcgctgcccagccggcgatggc

SEQ ID NO: 21 - protein, 272 aa, Artificial Sequence
  Synthetic peptide sequence
MKYLLPTAAAGLLLLAAQPAMAMANIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIKEGKSSGSGSESKVDEVQLQQSGPELVKPGTSVKISCKTSGYTFTEYTIHWVKQSHGKSLAWIGGIDPNSGGTNYSPNFKGKATLTVDKSSSTAYMDLRSLSSEDSAVYFCARIYHYDGYFDVWGAGTAVTVSSLEHHHHHH

SEQ ID NO: 22 - protein, 329 aa, Artificial Sequence
  Synthetic peptide sequence
MKYLLPTAAAGLLLLAAQPAMAMANIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIKEGKSSGSGSESKVDEVQLQQSGPELVKPGTSVKISCKTSGYTFTEYTIHWVKQSHGKSLAWIGGIDPNSGGTNYSPNFKGKATLTVDKSSSTAYMDLRSLSSEDSAVYFCARIYHYDGYFDVWGAGTAVTVSSGGGSGGGSGGGSGGGEDPCACESLVKFQAKVEGLLQALTRKLEAVSKRLAILENTVVLEHHHHHH

SEQ ID NO: 23 - protein, 507 aa, Artificial Sequence
  Synthetic peptide sequence
MKYLLPTAAAGLLLLAAQPAMAMANIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIKEGKSSGSGSESKVDEVQLQQSGPELVKPGTSVKISCKTSGYTFTEYTIHWVKQSHGKSLAWIGGIDPNSGGTNYSPNFKGKATLTVDKSSSTAYMDLRSLSSEDSAVYFCARIYHYDGYFDVWGAGTAVTVSSGGGEPKSSDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLEHHHHHH

SEQ ID NO: 24 - protein, 22 aa, Erwinia carotovora
MKYLLPTAAAGLLLLAAQPAMA

SEQ ID NO: 25 - protein, 107 aa, Mus musculus
NIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIK

SEQ ID NO: 26 - protein, 14 aa, Artificial Sequence
  Synthetic peptide sequence
EGKSSGSGSESKVD

SEQ ID NO: 27 - protein, 119 aa, Mus musculus
EVQLQQSGPELVKPGTSVKISCKTSGYTFTEYTIHWVKQSHGKSLAWIGGIDPNSGGTNYSPNFKGKATLTVDKSSSTAYMDLRSLSSEDSAVYFCARIYHYDGYFDVWGAGTAVTVSS

SEQ ID NO: 28 - protein, 15 aa, Artificial Sequence
  Synthetic peptide sequence
GGGSGGGSGGGSGGG

SEQ ID NO: 29 - protein, 75 aa, Artificial Sequence
  Synthetic peptide sequence
LVTFKNPHAKKQDVVVGGKLTNTTTESRCPTQGEPSLNEEQDKRFLCKHSMVDRGWGNGCGLFGKGGIVTCAMFT

SEQ ID NO: 30 - protein, 75 aa, Artificial Sequence
  Synthetic peptide sequence
LVTFKTAHAKKQEVVVGGKISNTTTDSRCPTQGEATLVEEQDTNFVCRRTFVDRGWGNGCGLFGKGSLITCAKFK

SEQ ID NO: 31 - protein, 75 aa, Artificial Sequence
  Synthetic peptide sequence
LVTFKNAHAKKQEVVVGGKITNITTDSRCPTQGEAILPEEQDQNYVCKHTYVDRGWGNGCGLFGKGSLVTCAKFQ

SEQ ID NO: 32 - protein, 75 aa, Artificial Sequence
  Synthetic peptide sequence
MVTFKVPHAKRQDVTVGGSISNITTATRCPTQGEPYLKEEQDQQYICRRDVVDRGWGNGCGLFGKGGVVTCAKFS

SEQ ID NO: 33 - protein, 75 aa, Artificial Sequence
  Synthetic peptide sequence
LMEFEEPHATKQSVVAGGSVSDLSTRAACPTMGEAHNEKRADPAFVCKQGVVDRGWGNGCGLFGKGSIDTCAKFA

SEQ ID NO: 34 - protein, 75 aa, Artificial Sequence
  Synthetic peptide sequence
LMEFEGAHATKQSVVAGGSVTDISTVARCPTTGEAHNEKRADSSYVCKQGFTDRGWGNGCGLFGKGSIDTCAKFS

SEQ ID NO: 35 - protein, 75 aa, Artificial Sequence
  Synthetic peptide sequence
LVEFEEPHATKQSVVAGGTVSDVSTVSNCPTTGESHNTKRADHNYLCKRGVTDRGWGNGCGLFGKGSIDTCAKFT

SEQ ID NO: 36 - protein, 75 aa, Artificial Sequence
  Synthetic peptide sequence
LMEFEEPHATKQSVIAGGTVSELSTKAACPTMGEAHNDKRADPSFVCKQGVVDRGWGNGCGLFGKGSIDTCAKFA

SEQ ID NO: 37 - protein, 75 aa, Artificial Sequence
  Artificial peptide sequence
LVEFEEPHATKQTVVAGGTLDTLSTVARCPTTGEAHNTKRSDPTFVCKRDVVDRGWGNGCGLFGKGSIDTCAKFT

SEQ ID NO: 38 - protein, 75 aa, Artificial Sequence
  Synthetic peptide sequence
LVEFEPPHAATIRVLAGGVLTHVKINDKCPSTGEAHLAEENEGDNACKRTYSDRGWGNGCGLFGKGSIVACAKFT

SEQ ID NO: 39 - protein, 75 aa, Artificial Sequence
  Synthetic peptide sequence
LVEFGAPHAVKMDVYNGGKLSDTKVAARCPTMGPATLAEEHQSGTVCKRDQSDRGWGNHCGLFGKGSIVTCVKAS

SEQ ID NO: 40 - protein, 75 aa, Artificial Sequence
  Synthetic peptide sequence
LVEFGTPHAVKMDVFNGGKLTGTKVAARCPTMGPATLPEEHQSGTVCKRDQSDRGWGNHCGLFGKGSIVTCVKFT

SEQ ID NO: 41 - protein, 75 aa, Artificial Sequence
  Synthetic peptide sequence
LVEFGAPHAVKMDVYNGGKLSETKVAARCPTMGPAALAEERQIGTVCKRDQSDRGWGNHCGLFGKGSIVACVKAA

SEQ ID NO: 42 - protein, 75 aa, Artificial Sequence
  Synthetic peptide sequence
LVEFGPPHAVKMDVFNGGKLTNTKVEARCPTTGPATLPEEHQANMVCKRDQSDRGWGNHCGFFGKGSIVACAKFE

SEQ ID NO: 43 - protein, 75 aa, Artificial Sequence
  Synthetic peptide sequence
DTVYRNEHNKKHDVVSASKLTNTTTESRCTGQGVATLNEDEDKRFNCYLDLVYRGWGNGCGDRGLGFIKQCSMKV

SEQ ID NO: 44 - protein, 75 aa, Artificial Sequence
  Synthetic peptide sequence
DTEIYNEHGKKTDVVTTAKLTNTTTESRCPGQGEQTLNEDADQRFFCVKDLVYRGWGNGCGVRGWGTIQQCVMKV

SEQ ID NO: 45 - protein, 75 aa, Artificial Sequence
  Synthetic peptide sequence
DTVVRTEHKKKIDVVSSTKLTNTTTESRCPGQGESTLNEEEDERFDCQQDQVLRGWGNGCGVPGWGNIKKCAMKE

SEQ ID NO: 46 - protein, 75 aa, Artificial Sequence
  Synthetic peptide sequence
ESVSVNEHKKKNDVVSDTKLTNTTTESRCPGQGEPTLNEEEDERFDCQKDLVYRGWGNGCGVRGWGWIKKCAMKV

SEQ ID NO: 47 - protein, 75 aa, Artificial Sequence
  Synthetic peptide sequence
KVVSRNEHRKKNDVVSETKLTNTTTESRCPNQGEATLNEDEDERFECVKDVVYRGWGNGCGERGLGTIQQCWMNE

SEQ ID NO: 48 - protein, 75 aa, Artificial Sequence
  Synthetic peptide sequence
DDEYYLEHYKKLDVVSRTKLTNTTTESRCPGQGQATLNEEEDERFQCFVALVIRGWGNGCGVVGWGSIVVCKMKI

SEQ ID NO: 49 - protein, 75 aa, Artificial Sequence
  Synthetic peptide sequence
DTVVVNEHNKKIDVVSTSKLTNTTTESRCPGQGPATLNEAEDERFDCQFSYVYRGWGNGCGKRGLGPILVCAMKV

SEQ ID NO: 50 - protein, 75 aa, Artificial Sequence
  Synthetic peptide sequence
DNVVVNEHYKKTDVVSSTKLTNTTTESRCPGQGYPTLNEQSDERFVCFVDYVIRGWGNGCGVDGWGPIVVCKMKI

SEQ ID NO: 51 - protein, 42 aa, Homo sapiens
EDPCACESLVKFQAKVEGLLQALTRKLEAVSKRLAILENTVV

SEQ ID NO: 52 - protein, 232 aa, Homo sapiens
EPKSSDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 250 aa, Artificial Sequence
  Synthetic peptide sequence
MANIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIKEGKSSGSGSESKVDEVQLQQSGPELVKPGTSVKISCKTSGYTFTEYTIHWVKQSHGKSLAWIGGIDPNSGGTNYSPNFKGKATLTVDKSSSTAYMDLRSLSSEDSAVYFCARIYHYDGYFDVWGAGTAVTVSSLEHHHHHH

SEQ ID NO: 54 - protein, 307 aa, Artificial Sequence
  Synthetic peptide sequence
MANIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIKEGKSSGSGSESKVDEVQLQQSGPELVKPGTSVKISCKTSGYTFTEYTIHWVKQSHGKSLAWIGGIDPNSGGTNYSPNFKGKATLTVDKSSSTAYMDLRSLSSEDSAVYFCARIYHYDGYFDVWGAGTAVTVSSGGGSGGGSGGGSGGGEDPCACESLVKFQAKVEGLLQALTRKLEAVSKRLAILENTVVLEHHHHHH

SEQ ID NO: 55 - protein, 485 aa, Artificial Sequence
  Synthetic peptide sequence
MANIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIKEGKSSGSGSESKVDEVQLQQSGPELVKPGTSVKISCKTSGYTFTEYTIHWVKQSHGKSLAWIGGIDPNSGGTNYSPNFKGKATLTVDKSSSTAYMDLRSLSSEDSAVYFCARIYHYDGYFDVWGAGTAVTVSSGGGEPKSSDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLEHHHHHH

SEQ ID NO: 56 - protein, 479 aa, Artificial Sequence
  Synthetic peptide sequence
MANIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIKEGKSSGSGSESKVDEVQLQQSGPELVKPGTSVKISCKTSGYTFTEYTIHWVKQSHGKSLAWIGGIDPNSGGTNYSPNFKGKATLTVDKSSSTAYMDLRSLSSEDSAVYFCARIYHYDGYFDVWGAGTAVTVSSGGGEPKSSDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLE

(19) United States
(12) Patent Application Publication
(10) Pub. No.: US 2009/0312190 A1
(43) Pub. Date: Dec. 17, 2009

(54) Methods and Proteins for the Prophylactic and/or Therapeutic Treatment of Four Serotypes of Dengue Virus and Other Flaviviruses

(75) Inventors: Glay Chinea Santiago, Ciudad de La Habana (CU); Vivian Huerta Galindo, Ciudad de La Habana (CU); Alejandro Miguel Martin Dunn, Ciudad de La Habana (CU); Jorge Victor Gavilondo Cowley, Ciudad de La Habana (CU); Noralvis Fleitas Salazar, Ciudad de La Habana (CU); Osmany Guirola Cruz, Ciudad de La Habana (CU); Jeovanis Gil Valdés, Ciudad de La Habana (CU); Aída Zulueta Morales, Ciudad de La Habana (CU); Lisset Hermida Cruz, Ciudad de La Habana (CU); Marta Ayala Avila, Ciudad de La Habana (CU); Diamilé Gonzáles Roche, Ciudad de La Habana (CU); Rolando Paez Meireles, Ciudad de La Habana (CU); Patricia Gabriela Toledo Mayora, Ciudad de La Habana (CU); Mónica Sarria Núnez, Ciudad de La Habana (CU); Alexis Musacchio Lasa, La Habana (CU); Yuliet Mazola Reyes, La Habana (CU)

Correspondence Address: HOFFMANN & BARON, LLP, 6900 JERICHO TURNPIKE, SYOSSET, NY, 11791, US

(73) Assignee: CENTRO DE INGENIERÍA GENÉTICA Y BIOTECNOLOGÍA, Cubanacán, Playa (CU)

(21) Appl. No.: 12/094,503

(22) PCT Filed: Nov. 21, 2006
(86) PCT No.: PCT/CU2006/000015

§ 371 Date: Jan. 7, 2009

(30) Foreign Application Priority Data
Nov. 22, 2005 (CU) 2005-0229

(51) Int. Cl. C40B 30/02; A61K 39/00; A61K 39/12; C12N 7/00; C12N 5/00; C40B 30/04; C40B 40/10; C07K 16/00; C07H 21/04; A61K 39/02

(52) U.S. Cl. 506/8; 424/185.1; 424/204.1; 435/235.1; 435/325; 506/9; 506/18; 530/350; 530/387.1; 536/23.1

(57) ABSTRACT

The present invention is related to the field of the pharmaceutical industry, and describes a conserved area on the surface of the E protein that can be used for the development of wide-spectrum antiviral molecules to be employed in the prophylaxis and/or treatment of infections due to Dengue Virus serotypes 1-4 and other flaviviruses. The invention also covers chimeric proteins to be used as vaccines or as a prophylactic or therapeutic treatment against the four serotypes of Dengue Virus and other flaviviruses.

### FIELD OF THE INVENTION

The present invention is related to the field of the pharmaceutical industry, and describes a conserved area on the surface of the E protein that can be used for the development of wide-spectrum antiviral molecules to be employed in the prophylaxis and/or treatment of infections due to Dengue Virus serotypes 1-4 and other flaviviruses. The invention describes methods and proteins useful for the prophylactic and/or therapeutic treatment of the four serotypes of Dengue Virus and, alternatively, other flaviviruses.

### PREVIOUS ART

The Dengue Virus (DV) complex belongs to the Flaviviridae family, and is composed of four different viruses or serotypes (DV1-DV4), genetically and antigenically related. DV is transmitted to man through mosquitoes, mainly Aedes aegypti. The infection produces varying clinical symptoms, ranging from asymptomatic and benign manifestations such as undifferentiated febrile episodes to more severe manifestations like Dengue Hemorrhagic Fever (DHF) and the life-threatening Dengue Shock Syndrome (DSS). The more severe clinical symptoms are usually associated to sequential infections with two different serotypes (Halstead, S. B. Neutralization and antibody-dependent enhancement of dengue viruses. Adv. Virus Res. 60:421-67., 421-467, 2003. Hammon W Mc. New haemorragic fever in children in the Philippines and Thailand. Trans Assoc Physicians 1960; 73: 140-155), a finding that has been corroborated by several epidemiological studies (Kourí G P, Guzmán M G, Bravo J R. Why dengue hemorrhagic fever in Cuba? 2. An integral analysis. Trans Roy Soc Trop Med Hyg 1987; 72: 821-823). This phenomenon has been explained by the theory of antibody-dependant enhancement (ADE), which argues that, in these cases, there is an increase in viral infectivity due to an increase in the entry of virus-antibody complexes to their target (the monocytes), mediated by the Fc receptors present on these cells (Halstead S B. Pathogenesis of dengue: challenges to molecular biology. Science 1988; 239: 476-481).

The envelope glycoprotein (E-protein) is the largest structural protein of the viral envelope. The three-dimensional structures of a fragment of the ectodomain of E-protein from DEN2 and DEN3 viruses have recently been solved by x-ray diffraction techniques (Modis, Y., Ogata, S., Clements, D. & Harrison, S. C. A ligand-binding pocket in the dengue virus envelope glycoprotein. Proc. Natl. Acad. Sci. U.S.A 100, 6986-6991,2003. Modis, Y., Ogata, S., Clements, D., and Harrison, S. C. Variable Surface Epitopes in the Crystal Structure of Dengue Virus Type 3 Envelope Glycoprotein. J. Virol. 79, 1223-1231, 2005), showing a high degree of structural similarity to the crystal structure of E-protein from Tick-borne Encephalitis Virus (Rey F. A., Heinz, F. X., Mandl, C., Kunz, C. & Harrison, S. C. The envelope glycoprotein from tick-borne encephalitis virus at 2 A resolution. Nature 375, 291-298, 1995). This structural similarity is congruent with their sequence homology, the conservation of 6 disulphide bridges, and the conservation of the localization of residues to which a functional role, such as being part of an antigenic determinant or being involved in attenuation or escape mutations, has been previously assigned in other flaviviruses.

Protein E is formed by three structural domains: domain I, located on the N-terminal part of the sequence but forming the central domain in the 3D structure; domain II, also known as the dimerization domain, which contains a fusion peptide highly conserved across flaviviruses; and domain III, with an immunoglobulin-like fold, which is involved in the interaction with cellular receptors.

Protein E is a multifunctional glycoprotein that plays a central role in several stages of the viral life cycle. This protein is the main target for virus-neutralizing antibodies, mediates the interaction with the cellular receptors, and is the engine driving the fusion between the viral and cellular membranes (Heinz, F. X., and S. L. Allison. 2003. Flavivirus structure and membrane fusion. Adv. Virus Res. 59:63-97. Modis, Y., S. Ogata, D. Clements, and S. C. Harrison. 2004. Structure of the dengue virus envelope protein after membrane fusion. Nature 427:313-319. Rey 2004. Chen, Y., T. Maguire, R. E. Hileman, J. R. Fromm, J. D. Esko, R. J. Linhardt, and R. M. Marks. 1997. Dengue virus infectivity depends on envelope protein binding to target cell heparan sulfate. Nat. Med. 3:866-871. Navarro-Sanchez, E., R. Altmeyer, A. Amara, O. Schwartz, F. Fieschi, J. L. Virelizier, F. Arenzana-Seisdedos, and P. Despres. 2003. Dendritic-cell-specific ICAM3-grabbing non-integrin is essential for the productive infection of human dendritic cells by mosquito-cell-derived dengue viruses. EMBO Rep. 4:1-6. Tassaneetrithep, B., T. H. Burgess, A. Granelli-Piperno, C. Trumpfheller, J. Finke, W. Sun, M. A. Eller, K. Pattanapanyasat, S. Sarasombath, D. L. Birx, R. M. Steinman, S. Schlesinger, and M. A. Marovich. 2003. DC-SIGN (CD209) mediates dengue virus infection of human dendritic cells. J. Exp. Med. 197:823-829).

This protein is anchored to the viral membrane, and its functions are associated to large conformational changes, both in tertiary and quaternary structure. During the intracellular stages of virus formation, E is found as a heterodimer together with the preM protein (Allison, S. L., K. Stadler, C. W. Mandl, C. Kunz, and F. X. Heinz. 1995. Synthesis and secretion of recombinant tick-borne encephalitis virus protein E in soluble and particulate form. J. Virol. 69:5816-5820. Rice, C. M. 1996. Flaviviridae: the viruses and their replication, p. 931-959. In B. N. Fields, D. N. Knipe, P. M. Howley, R. M. Chanock, J. L. Melnick, T. P. Monath, B. Roizman, and S. E. Straus (ed.), Virology, 3rd ed. Lippincott-Raven, Philadelphia, Pa.). During this stage the virions are said to be immature, and are defective for mediating membrane fusion, as evidenced in their dramatically lower infectivity in vitro when compared to mature extracellular virions (Guirakhoo, F., Heinz, F. X., Mandl, C. W., Holzmann, H. & Kunz, C. Fusion activity of flaviviruses: comparison of mature and immature (prM containing) tick-borne encephalitis virions. J. Gen. Virol. 72, 1323-1329, 1991. Guirakhoo, F., Bolin, R. A. & Roehrig, J. T. The Murray Valley encephalitis virus prM protein confers acid resistance to virus particles and alters the expression of epitopes within the R2 domain of E glycoprotein. Virology 191, 921-931,1992). It is postulated that the role of the heterodimers is to prevent the binding of protein E to the membrane during the traffic of the virions through intracellular compartments that could, due to their acidic pH, trigger the membrane fusion process. Besides, it is possible that the preM protein functions as a chaperone for the folding and assembly of protein E (Lorenz, I. C., Allison, S. L., Heinz, F. X. & Helenius, A. Folding and dimerization of tick-borne encephalitis virus envelope proteins prM and E in the endoplasmic reticulum. J. Virol. 76, 5480-5491, 2002). During secretion of the virions out of the cell, preM is enzymatically processed by host proteases (furins), leaving E free to associate as homodimers and therefore triggering a reorganization of the viral envelope that ends with the formation of mature virions (Stadler, K., Allison, S. L., Schalich, J. & Heinz, F. X. Proteolytic activation of tick-borne encephalitis virus by furin. J. Virol. 71, 8475-8481, 1997. Elshuber, S., Allison, S. L., Heinz, F. X. & Mandl, C. W. Cleavage of protein prM is necessary for infection of BHK-21 cells by tick-borne encephalitis virus. J. Gen. Virol. 84, 183-191, 2003). The structure of mature virions has been determined by electronic cryomicroscopy at a resolution of 9.5 Å (Zhang W, Chipman P R, Corver J, Johnson P R, Zhang Y, Mukhopadhyay S, Baker T S, Strauss J H, Rossmann M G, Kuhn R J. Visualization of membrane protein domains by cryo-electron microscopy of dengue virus. Nat Struct Biol. 2003, 10: 907-12. Kuhn, R. J. et al. Structure of dengue virus: implications for flavivirus organization, maturation, and fusion. Cell 108, 717-725, 2002), and that of immature virions, at 12.5 Å (Zhang, Y. et al. Structures of immature flavivirus particles. EMBO J. 22, 2604-2613, 2003). These virions have a T=3 icosahedral symmetry. In the mature virions the protein E dimers lay on a plane parallel to the viral membrane, covering its surface almost completely. Mature virions are completely infective, and it is in this conformation that the virus interacts with the cellular receptors and the antibodies elicited by the host. Once the virus engages the cellular receptors, it is internalized through receptor-mediated endocytosis, and eventually reaches the endosomes, where the slightly acid pH triggers the conformational change in protein E that starts the membrane fusion process (Allison, S. L. et al. Oligomeric rearrangement of tick-borne encephalitis virus envelope proteins induced by an acidic pH. J. Virol. 69, 695-700, 1995). In this process the protein reorganizes from dimers to trimers. The post-fusogenic structure of protein E has been recently determined (Modis, Y., Ogata, S., Clements, D. & Harrison, S. C. Structure of the dengue virus envelope protein after membrane fusion. Nature 427, 313-319 (2004). Bressanelli, S. et al. Structure of a flavivirus envelope glycoprotein in its low-pH-induced membrane fusion conformation. EMBO J. 23, 728-738 (2004), showing that trimer formation involves important rearrangements in tertiary structure, with the monomers associating in parallel while the tip of domain II, containing the fusion peptide, interacts with the membranes. By analyzing together the crystal structures and the resolved virion structures it becomes evident that throughout the viral life cycle protein E is subjected to rearrangements in which its ternary and quaternary structure, as well as the virion itself, changes dramatically.

Protein E is the main target of the neutralizing antibodies generated during the viral infection. An infection with a single serotype elicits long-lived antibodies which are neutralizing against viruses of the homologous serotype. During the first months after the infection these antibodies can neutralize heterologous serotypes as well, but this activity slowly decreases until it disappears, about 9 months post-infection (Halstead S. B. Neutralization and antibody-dependent enhancement of dengue viruses. Adv Virus Res. 2003;60: 421-67. Sabin, A. B. 1952. Research on dengue during World War II. Am. J. Trop. Med. Hyg. 1: 30-50.)

The antibodies generated against one serotype generally display a decreased affinity when interacting with viruses of a different serotype; a phenomenon that is explained at the molecular level by variations in the aminoacid sequence of protein E among DV serotypes. An interaction of sufficiently low affinity can result in an antibody that fails to neutralize, but is still able to bind the viral surface in amounts enough to facilitate the internalization of the virus into cells carrying Fc receptors (Halstead, S. B., and E J. O'Rourke. 1977. Dengue viruses and mononuclear phagocytes. I. Infection enhancement by non-neutralizing antibody. J. Exp. Med. 146:201-217. Littaua, R., I. Kurane, and F. A. Ennis. 1990. Human IgG Fc receptor II mediates antibody-dependent enhancement of dengue virus infection. J. Immunol. 144:3183-3186).

Additionally, during a secondary infection the titer of the low-affinity antibody population surpasses that of the new high-affinity antibodies generated by the incoming DV serotype, due to the faster activation of pre-existing memory B-cells and plasma cells when compared to naive B-cells. The antibody profile during convalescence from secondary infections is greatly influenced by the serotype of the primary DV infection, a fact that is just a manifestation of the phenomenon known as “original antigenic sin” (Halstead, S. B., Rojanasuphot, S., and Sangkawibha, N. 1983. Original antigenic sin in dengue. Am. J. Trop. Med. Hyg. 32:154-156).

On the other hand, it is known that highly potent neutralizing monoclonal antibodies (mAbs) can trigger immunoamplification at high dilutions (Brandt, W. E., J. M. McCown, M. K. Gentry, and P. K. Russell. 1982. Infection enhancement of dengue type 2 virus in the U-937 human monocyte cell line by antibodies to flavivirus cross-reactive determinants. Infect. Immun. 36:1036-1041. Halstead, S. B., C. N. Venkateshan, M. K. Gentry, and L. K. Larsen. 1984. Heterogeneity of infection enhancement of dengue 2 strains by monoclonal antibodies. J. Immunol. 132:1529-1532. Morens, D. M., S. B. Halstead, and N. J. Marchette. 1987. Profiles of antibody-dependent enhancement of dengue virus type 2 infection. Microb. Pathog. 3:231 237).

The antigenic structure of the flaviviral protein E has been intensely studied, using murine mAb panels and a group of biochemical and biological analyses that includes competition assays, sensitivity of the interaction to procedures such as reduction of the disulphide bridges and treatment with SDS, assays for binding to proteolytic fragments and synthetic peptides, assays for viral neutralization or inhibition of hemagglutination, generation of escape mutants, serological tests, etc. (Heinz. T. Roehrig, J. T., Bolin, R. A. and Kelly, R. G. Monoclonal Antibody Mapping of the Envelope Glycoprotein of the Dengue 2 Virus, Jamaica, VIROLOGY 246, 317-328, 1998 Heinz, F. X., and Roehrig, J. T. (1990). Flaviviruses. In “Immunochemistry of Viruses. II. The Basis for Serodiagnosis and Vaccines” (M. H. V. Van Regenmortel and A. R. Neurath, Eds.), pp. 289-305. Elsevier, Amsterdam. Mandl, C. W., Guirakhoo, F. G., Holzmann, H., Heinz, F. X., and Kunz, C. (1989). Antigenic structure of the flavivirus envelope protein E at the molecular level, using tick-borne encephalitis virus as a model. J. Virol. 63, 564-571. I. L. Serafin and J. G. Aaskov. Identification of epitopes on the envelope (E) protein of dengue 2 and dengue 3 viruses using monoclonal antibodies. Arch Virol (2001) 146: 2469-2479. Three antigenic domains, A, B and C, have been defined, which correspond to the three structural domains II, III and I, respectively. The antibodies recognizing a particular epitope usually show very similar functional characteristics. The recognition of epitopes from domain A (equivalent to structural domain II) is destroyed by the reduction of disulphide bridges, and the mAbs recognizing these epitopes inhibit hemagglutination, neutralize viral infection and inhibit virus-mediated membrane fusion. Particularly, epitope A1, defined for Dengue Virus, is recognized by mAbs with group-type specificity, i.e. they are highly cross-reactive among different flaviviruses. The mAbs 4G2 (anti-DV2) and 6B6C (anti-JEV) recognize this epitope. Binding to this epitope is diminished by several orders of magnitude in immature virions, and is not enhanced by acid pH treatment of mature virions (Guirakhoo, F., R. A. Bolin, and J. T. Roehrig. 1992. The Murray Valley encephalitis virus prM protein confers acid resistance to virus particles and alters the expression of epitopes within the R2 domain of E glycoprotein. Virology 191:921-931).

Vaccine Development

No specific treatments against DV and its most severe manifestations are currently available. Mosquito control is costly and not very efficient. Although the clinical treatments based on a proper management of fluids to correct the hypovolemia caused by DHF has decreased its mortality, these treatments are still problematic in many underdeveloped nations. It has been estimated that 30 000 deaths per year are attributable to DHF, and the morbility and associated costs of this disease are comparable to those of other diseases which constitute first priority targets of public health spending (Shepard D S, Suaya J A, Halstead S B, Nathan M B, Gubler D J, Mahoney R T, Wang D N, Meltzer M I. Cost-effectiveness of a pediatric dengue vaccine. Vaccine. 2004, 22(9-10):1275-80).

Several vaccine candidates against dengue are currently in different stages of development (Barrett, A. D. 2001. Current status of flavivirus vaccines. Ann. N.Y. Acad. Sci. 951:262-271. Chang G J, Kuno G, Purdy D E, Davis B S. 2004 Recent advancement in flavivirus vaccine development. Expert Rev Vaccines. 2004 3(2):199-220 ). The strategies tried so far include attenuated live vaccines, chimeric viruses, plasmid DNA and subunit vaccines. Attenuated strains from the four serotypes have been developed using standard methodologies for viral propagation in primary kidney cells of dogs and monkeys (Bhamarapravati, N., and Sutee, Y. 2000. Live attenuated tetravalent dengue vaccine. Vaccine. 18:44-47. Eckels, K. H., et al. 2003. Modification of dengue virus strains by passage in primary dog kidney cells: preparation of candidate vaccines and immunization of monkeys. Am. J. Trop. Med. Hyg. 69:12-16. Innis, B. L., and Eckels, K. H. 2003. Progress in development of a live-attenuated, tetravalent dengue virus vaccine by the United States Army Medical Research and Materiel Command. Am. J. Trop. Med. Hyg. 69:1-4). The advance of this strategy has been limited by the lack of animal models and in vitro markers of attenuation for humans. In this same research avenue, there is work in which cDNA clones have been obtained from the four serotypes and have then been treated to introduce attenuating mutations and variations that, in theory, greatly decrease the possibility of reversion to virulent phenotypes (Blaney, J. E., Jr., Manipon, G. G., Murphy, B. R., and Whitehead, S. S. 2003. Temperature sensitive mutations in the genes encoding the NS1, NS2A, NS3, and NS5 nonstructural proteins of dengue virus type 4 restrict replication in the brains of mice. Arch. Virol. 148:999-1006. Durbin, A. P., et al. 2001. Attenuation and immunogenicity in humans of a live dengue virus type-4 vaccine candidate with a 30 nucleotide deletion in its 3′-untranslated region. Am. J. Trop. Med. Hyg. 65:405-413. Patent: Zeng L, Markoff L, WO0014245, 1999)

Another strategy has been the creation of chimeric flaviviral variants for the four serotypes, introducing the preM and E structural proteins from one dengue serotype into an attenuated background of Yellow Fever Virus (YFV), dengue or other virus that contribute the Core and other non-structural proteins (Guirkhoo F, Arroyo J, Pugachev K V et al. Construction, safety, and immunogenicity in non-human primates of a chimeric yellow fever-dengue virus tetravalent vaccine. J Virol 2001; 75: 7290-304. Huang C Y, Butrapet S, Pierro D J et al. Chimeric dengue type 2 (vaccine strain PDK-53)/dengue type 1 virus as a potential candidate dengue type 1 virus vaccine. J Virol 2000; 74: 3020-28. Markoff L, Pang X, Houng H S, et al. Derivation and characterization of a dengue 1 host-range restricted mutant virus that is attenuated and highly immunogenic in monkeys. J Virol 2002; 76: 3318-28. Patent: Stockmair and schwan Hauesser, WO9813500, 1998. Patent: Clark and Elbing: WO9837911, 1998. Patent: Lai C J, U.S. Pat. No. 6,184,024, 1994.)

In general, multiple questions persist about the potential benefits of live attenuated vaccines, given the possibility of occurrence of phenomena such as reversions to virulent phenotypes, viral interference and intergenomic recombination (Seligman S J, Gould E A 2004 Live flavivirus vaccines: reasons for caution. Lancet. 363(9426):2073-5). The vaccines based on plasmid DNA expressing recombinant proteins are still in the early stages of development, as well as those based in recombinant antigens (Chang, G. J., Davis, B. S., Hunt, A. R., Holmes, D. A., and Kuno, G. 2001. Flavivirus DNA vaccines: current status and potential. Ann. N.Y. Acad. Sci. 951:272-285. Simmons, M., Murphy, G. S., Kochel, T., Raviprakash, K., and Hayes, C. G. 2001. Characterization of antibody responses to combinations of a dengue-2 DNA and dengue-2 recombinant subunit vaccine. Am. J. Trop. Med. Hyg. 65:420-426. Patent: Hawaii Biotech Group, Inc; WO9906068 1998. Feighny, R., Borrous, J. and Putnak R. Dengue type-2 virus envelope protein made using recombinant baculovirus protects mice against virus challenge. Am. J. Trop. Med. Hyg. 1994. 50(3). 322-328; Deubel, V., Staropol I., Megret, F., et al. Affinity-purified dengue-2 virus envelope glycoprotein induces neutralising antibodies and protective immunity in mice. Vaccine. 1997. 15, 1946-1954)

Several vaccine candidates based on the strategies described above have showed protection in animal models, and some have been found to be safe and immunogenic during the early stages of clinical trials.

The main hurdle for vaccine development, however, is the need for achieving equally effective protection against the four serotypes. It is agreed that an infection with one dengue serotype induces lifelong immunity against the same serotype in humans. However, immunizing against only one serotype achieves protection against other serotypes (heterotypic immunity) only for a short period of time ranging from 2 to 9 months (Sabin, A. B. 1952. Research on dengue during World War II. Am. J. Trop. Med. Hyg. 1:30-50). Besides, a suboptimal level of protection against a specific serotype might sensitize the vacinee and increase the risk of appearance of severe manifestations associated to a heterologous immune response of a pathological nature, upon later infection with that serotype (Rothman A L 2004 Dengue: defining protective versus pathologic immunity J. Clin. Invest. 113:946-951). However, the development of effective tetravalent formulations of the available live attenuated or recombinant subunit vaccines has turned out to be a difficult challenge, requiring the use of complicated, multi-dose immunization schedules.

Antibodies: Passive Immunization

One alternative to the use of vaccines for the prevention of dengue infection is the use of neutralizing antibodies for passive immunization. Humanized chimpanzee antibodies have been obtained for this purpose, including 5H2, which neutralizes dengue 4 (Men, R., T. Yamashiro, A. P. Goncalvez, C. Wernly, D. J. Schofield, S. U. Emerson, R. H. Purcell, and C. J. Lai. 2004. Identification of chimpanzee Fab fragments by repertoire cloning and production of a full-length humanized immunoglobulin G1 antibody that is highly efficient for neutralization of dengue type 4 virus. J. Virol. 78:4665-4674), and 1A5, which is cross-neutralizing against the four serotypes (Goncalvez, A. P., R. Men, C. Wernly, R. H. Purcell, and C. J. Lai. 2004. Chimpanzee Fab fragments and a derived humanized immunoglobulin G1 antibody that efficiently cross-neutralize dengue type 1 and type 2 viruses. J. Virol. 78: 12910-12918).

The use of passive immunization might be useful both for prophylactic and therapeutic means, taking into account that the level of viremia is an important predictor for the severity of the disease (Wang, W. K. D. Y. Chao, C. L. Kao, H. C. Wu, Y. C. Liu, C. M. Li, S. C. Lin, J. H. Huang, and C. C. King. 2003. High levels of plasma dengue viral load during defervescence in patients with dengue haemorrhagic fever: implications for pathogenesis. Virology 305:330-338. Vaughn, D. W, Green, S., Kalayanarooj, S., Innis, B. L., Nimmannitya, S., Suntayakorn, S., Endy, T. P., Raengsakulrach, B., Rothman, A. L., Ennis, F. A. and Nisalak, A. Dengue Viremia Titer, Antibody Response Pattern, and Virus Serotype Correlate with Disease Severity. J. Infect. Dis. 2000;181:2-9). However, the administration of antibodies is not free of potential pitfalls. According to the antibody-dependant enhancement (ADE) theory, if the concentration of neutralizing antibodies decreases to subneutralizing levels, the virus-antibody immunocomplexes may amplify viral entry to the cells bearing Fc receptors, thus increasing the level of viral replication. Therefore, high antibody levels would be required to avoid endangering the patient for more severe manifestations of the disease.

One possible solution is the obtention of antibody molecules with an Fc modified in such a way that the interaction with its receptors is significantly decreased. In this sense, a particularly attractive strategy is the mutation of residues in the Fc that affect directly the interaction with FCγR-I, FCγR-II and FCγRIII but not with FCRn, since the latter is involved in antibody recycling and therefore is pivotal in determining the half-life of the antibody in vivo.

Another alternative is the identification of neutralizing antibodies incapable of mediating ADE. At least one antibody has been described with these characteristics (Patent: Bavarian Nordic Res. Inst. WO9915692, 1998), which neutralizes DV2 without mediating ADE in an in vitro model. However, there are no descriptions of similar antibodies against other serotypes, and there is no available data on the characterization of this type of antibodies in in vivo models. An additional obstacle is the fact that the available animal models do not reproduce faithfully the course and characteristics of the infection in humans.

Another strategy related to the use of antibodies is the obtention of bispecific complexes between anti-dengue and anti-erythrocyte complement receptor 1 antibodies. These heteropolymers would bind the virus to erythrocytes, therefore greatly increasing the rate of viral clearance from blood to tissues (Hahn C S, French O G, Foley P, Martin E N, Taylor R P. 2001. Bispecific monoclonal antibodies mediate binding of dengue virus to erythrocytes in a monkey model of passive viremia. J Immunol. 2001 166:1057-65.).

### DETAILED DESCRIPTION OF THE INVENTION

The invention describes how to obtain effective molecules for prophylactic and/or therapeutic treatment against the four serotypes of Dengue Virus and other flaviviruses, by using an area or epitope in the surface of E-protein (‘E’ for Envelope), which is highly conserved in flaviviruses, as a target for said molecules. When used for vaccine design, the invention allows the generation of a neutralizing and protective effect, which is of similar magnitude for all four Dengue Virus serotypes, by circumscribing the antibody response to this region of E-protein and therefore eliminating responses against more variable regions of this protein, which can elicit serotype- or subcomplex-specific neutralizing antibodies that can lead to immunoamplification during later infection with other serotypes. Since the described area of E-protein is a topographic epitope, the invention includes the design of mutations and stabilizing connections to guarantee the correct folding and secretion of the E-protein subdomain that includes the aforementioned epitope. When used for the development of agents for passive immunization with prophylactic or therapeutic purposes, the invention also defines recombinant molecules capable of binding two, three or multiple symmetric copies of this epitope on the surface of mature flaviviral virions, said recombinant molecules having neutralizing and protective characteristics which are superior to those of natural antibodies and/or their FAb fragments due to their higher avidity and better potential for interfering with the structural changes undergone by the virions during the early stages of the viral replication cycle.

In a first embodiment, the invention describes the design of recombinant proteins that reproduce the antigenic and structural features of the E-protein epitope mentioned above. One of the described recombinant proteins is recognized by a mouse monoclonal antibody capable of neutralizing all four serotypes of Dengue Virus that also recognizes other flaviviruses. The immunization with this chimeric recombinant protein induces an antibody response that is neutralizing and protective against the four Dengue Virus serotypes, as well as other flaviviruses. The invention describes a method for designing the chimeric recombinant protein in such a way that the E-protein domain containing the common flaviviral neutralizing epitope folds correctly. This epitope is topographic in nature, and therefore its antigenicity is dependant upon the 3D structure of the molecule. The molecules obtained with this invention can be used in the pharmaceutical industry for the obtention of vaccine preparations against Dengue Virus and other flaviviruses, as well as for the design of diagnostic systems containing these proteins.

The second embodiment of this invention describes the design of other recombinant proteins with a potent neutralizing profile against the four serotypes of Dengue Virus and other flaviviruses. The aminoacid sequence of these proteins contains a binding domain, a spacer segment, and a multimerization domain. The binding domain is capable of binding to an epitope of the E protein that is highly conserved across all flaviviruses, which is contained in the proteins described on the first object of this invention, described above. In a variant of this embodiment, the binding domains are single-chain antibody fragments that recognize the conserved epitope. The spacer segments are sequences 3-20 aminoacids long, enriched in residues which are preferably hydrophilic, polar and with a small side chain, therefore conferring the spacer a high degree of mobility. These spacers must not interfere with the folding of the binding and multimerization domains, and must additionally be resistant to cleavage by serum proteases.

The multimerization domains described in the present invention are proteins or protein domains that associate in their native state preferably as dimers or trimers, although quaternary structures of higher order of association are not discarded. These domains are selected from human serum or extracellular proteins, so as to avoid the possible induction of autoantibodies. An essential property of the multimerization domains considered in this invention is the absence of any interactions with Fc receptors, which are involved in the antibody-mediated process of immunoamplification of Dengue Virus infections. The quaternary structure of the multimerization domain may depend on covalent or non-covalent interactions.

In one of the variants, the multimerization domain is based on the Fc fragment from human antibodies, including the hinge region since it mediates the formation of inter-chain disulphide bridges that stabilize the dimeric structure. These Fc fragments are devoid of carbohydrate chains, either through chemical or enzymatic deglycosylation, or through their production on a host which does not glycosylate proteins, such as the bacterium Escherichia coli. The non-glycosylated Fc domains can also be obtained in cells from higher eukaryotes, provided that their sequence has been modified to remove the NXT/S motif. Non-glycosylated Fc domains can no longer bind to FcγR receptors I to III, which are mediators of immunoamplification in vitro. However, they remain fully competent for interacting with the FcRn receptor, which is a desirable property for obtaining a long half-life in vivo.

In another variant, the multimerization domain is a helicoidal, trimer-forming fragment of human matrilin.

The connection of the binding and multimerization domains through flexible spacers allows the simultaneous binding of the chimeric protein to multiple adjacent E-protein monomers on the icosahedral structure of flaviviral mature virions. This way, a sequence variant of [binding domain]-[spacer]-[multimerization domain] that yielded a dimeric protein would be able to bind simultaneously two E-protein monomers. Similarly, if the variant yields a trimeric protein, three monomers would be simultaneously bound.

The neutralization titer of the chimeric proteins described in the second embodiment of this invention is higher than that reached by FAb fragments and even complete antibodies. These recombinant proteins bind the virions with higher avidity, and the simultaneous engagement of several E monomers interferes with the necessary changes in quaternary structure during the process of membrane fusion. The molecules obtained with the practice of this invention can be used in the pharmaceutical industry for the obtention of prophylactic and/or therapeutic agents against Dengue Virus and other flaviviruses, as well as for the development of diagnostic systems containing said molecules.

Design of Chimerical Proteins for Vaccine Purposes

The currently accepted point of view is that an effective Dengue vaccine should induce a neutralizing antibody response against the four Dengue serotypes. However, the viral envelope E-glycoprotein is variable among the serotypes. This sequence variability cause that the global antibody response against the protein is neutralizing against the homolog serotype but not against the heterolog serotypes, this way increasing the possibility of rising infection immune-enhancing antibodies.

The current invention describes a method aimed at designing subunit vaccines against Dengue virus, which induce an immune response uniformly neutralizing and protective against the four serotypes. At first place the design is based on the identification of patches or epitopes exposed at the surface of the protein, which conservation is total or very high among serotypes and are also exposed on the surface of the mature virions. Carrying out a residue conservation analysis on the protein, it was possible to identify a cluster of exposed and conserved residues. (FIGS. 1 and 2, table 1).The total surface area of the cluster is 417 Å², belonging to 25 residues. This area is comparable with the typical values corresponding to the binding surface involved in protein-antibody interactions. The epitope is topographic, including residues located far apart on the primary structure of the protein, but close in the three dimensional structure.

At second place, the invention describes the design of recombinant chimerical proteins which contain the conserved epitope, maximizing the ratio between conserved/variable residues presented to the immune system and achieving the stabilization of the three dimensional structure of the epitope in a similar way as it appears in the context of the whole E-protein. Two possible topologies are described:
B-L-C and C-L-B,
   where B is the segment Leu237-Val252 and C is the segment Lys64-Thr120 of the E-glycoprotein from dengue 2 or the homolog segments from the other serotypes or other flavivirus, or similar protein sequences displaying more than 80% of sequence identity with respect to any of the above mentioned segments. The homolog segments B and C corresponding to flavivirus sequences are defined by the use of sequence alignment computer programs, which align pair of sequences or multiple sequences such like BLAST, FASTA y CLUSTAL (Altschul, S. F., Gish, W., Miller, W., Myers, E. W. & Lipman, D. J. 1990, Basic local alignment search tool. J. Mol. Biol. 215: 403-410. Pearson W R, Lipman D J. Improved tools for biological sequence comparison. Proc Natl Acad Sci USA. 1988;85:2444-8. Higgins D., Thompson J., Gibson T. Thompson J. D., Higgins D. G., Gibson T. J. 1994; CLUSTAL W: improving the sensitivity of progressive multiple sequence alignment through sequence weighting,position-specific gap penalties and weight matrix choice. Nucleic Acids Res. 22:4673-4680). These sequence alignments allow also define homolog residues (also referred as equivalent or corresponding residues) in the sequence of flavivirus, which correspond to the highly conserved residues identified in table 1 of the example 1 for the particular case of the sequence from dengue 2.

For both described topologies, L are linker sequences with a size of typically between 1 and 10 residues, whose role is to connect segments B and C in a stabilizing manner regarding the folding of the chimerical protein and allowing the 3D structure of the epitope to be similar to the structure displayed in the context of the whole E-protein. In both topological variants of the chimerical protein, the conserved epitope is completely included, excluding the rest of the E-protein which is more variable. The chimerical protein represents a sub-domain of the structural domain II of the envelope glycoprotein. This sub-domain is located at the tip of domain II and is structurally conformed by two anti-parallel beta sheets, packed against each other. The major beta sheet is composed by three beta strands (segment C) and the minor is a beta hair pin loop (segment B).

The sub-domain contains two disulfide bridges and it is connected to the rest of the E-glycoprotein through four points, which is consistent with the topographic nature of the conserved epitope. However, the contact surface between the sub-domain and the rest of the protein is 184 Å², which represents only the 12% of the total solvent accessible surface of the sub-domain. This fact is consistent with the feasibility to achieve the correct folding of the sub-domain by designing stabilizing connections or linkers as described above for the two topological variants. The invention includes the possibility of increasing the thermodynamic stability of the chimerical protein by means of mutations in residues which are not accessible to the virion surface and hence not involved in the interaction with antibodies.

An essential novelty of the present invention is the idea that it is possible to develop a subunit vaccine based on a unique protein chain, which is effective against the four Dengue serotypes. The current approaches based on recombinant protein candidates consist on the use of four recombinant envelope proteins, one for each serotype, which are combined in a vaccine formulation (Patente: Hawaii Biotech Group, Inc; WO9906068 1998). E-protein fragments have also been evaluated as possible candidates, but till now the efforts have been focused on domain III, expressed as fusion proteins with carrier proteins (Patente: Centro de Ingeniería Genética y Biotecnología; WO/2003/008571. Simmons M, Murphy G S, Hayes C G. Short report: Antibody responses of mice immunized with a tetravalent dengue recombinant protein subunit vaccine. Am J Trop Med Hyg. 2001;65:159-61. Hermida L, Rodriguez R, Lazo L, Silva R, Zulueta A, Chinea G, Lopez C, Guzman M G, Guillen G. A dengue-2 Envelope fragment inserted within the structure of the P64k meningococcal protein carrier enables a functional immune response against the virus in mice. J Virol Methods. January 2004;115(1):41-9). Domain III is able to induce a neutralizing antibody response, but this response is serotype specific and therefore a vaccine candidate should include sequences from the four serotypes.

The chimerical protein PMEC1 of the example 1 of the present invention corresponds to the topology B-L-C, with sequences of the fragment B and C from dengue 2 and a two residues Gly-Gly linker sequence. It is also described a gene which codifies for the chimerical protein PMEC1. The plasmid pET-sPMEC1-His6 codify for the protein PMEC1 fused at the N-terminus to the signal peptide pelB and at the C-terminus to a sequence codifying for six histidines (Sequence No. 12).

The chimerical protein PMEC1 was obtained soluble in the periplasm of the bacteria E. coli. An easily scalable purification process was developed based on metal chelates chromatography (IMAC), which allowed obtaining pure protein preparations suitable for further studies. The purified protein was analyzed by mass spectrometry and the obtained mass/z signal corresponds to the theoretical valued calculated from the amino acid sequence of PMEC1, assuming the formation of two disulfide bridges. The protein PMEC1 shows a strong recognition by hyper-immune ascitic fluids obtained against the four Dengue virus serotypes and by the mAb 4G2. This recognition depends on the correct formation of the disulfide bridges, suggesting that the protein PMEC1 has a conformation similar to the one adopted by the corresponding region of the native E-protein.

By immunizing mice with the chimerical protein PMEC1, it was obtained a neutralizing and protective response, characterized by high titers against the four Dengue serotypes.

An IHA assay was also performed and positive titers were obtained against the four serotypes. Titers of 1:1280 against the four serotypes were obtained in the in vitro neutralization test. Finally, a protection assay was carried out in mice, showing protection in 80-90% of animals against the four serotypes.

Modeling the Complex Formed by mAb 4G2 and the E-Protein

The example No. 8 shows the modeling of the structure of the complex formed by mAb 4G2 and the E-protein. This antibody recognizes and neutralizes the four Dengue serotypes and other flavivirus.

The model was obtained using the CLUSPRO protein-protein docking method (http://structure.bu.edu/Projects/PPDocking/cluspro.html). In the study, the crystallographic structure of FAb 4G2 (PDB file 1uyw) and the PDB files 1oan and 1oam corresponding to the dimeric structure of E-protein from dengue 2 were used as input files. The table 8 shows the values corresponding to the characteristic surface parameters of the E-protein-Fab interface, the values calculated for the modeled complex are similar to the typical values obtained for protein-antibody complexes which crystallographic structure has been solved (table 9).

The obtained model indicates that the epitope recognized by mAb 4G2, includes the highly conserved region identified in this invention. The table 1 shows the set of residues conforming the predicted structural epitope (residues making contacts with the antibody) and those residues belonging to the highly conserved surface patch. According to the model, the 71% of residues from the predicted structural epitope, belong to the highly conserved area.

Later, a model of the complex in the context of the mature virion was obtained by docking the previously predicted model on the cryo-electron microscopy structure of dengue 2 virus. This way, a model was obtained where all epitopes (180 copies) recognized by mAb 4G2 on the virion surface are occupied by FAb chains.

The inter-atomic distance between the C-terminus of the heavy chains corresponding to those FAbs bound to E-protein dimers is 100 Å. The same distance calculated for FAbs bound to monomers of the asymmetric unit, which are not associated as dimers, is 120 and 80 Å.

These distances are not stereo-chemically compatibles with the sequence and structure of the IgG molecule, suggesting that mAb 4G2 binds to the virus in a monovalent way.

This prediction is supported by the results shown in the example 12, which indicate that the FAb and the mAb 4G2 have very similar neutralizing titers. This finding contrast with data obtained for other antiviral antibodies, whose divalent binding causes an increase in the neutralizing capacity of 2-3 orders of magnitude (Drew P D, Moss M T, Pasieka T J, Grose C, Harris W J, Porter A J. Multimeric humanized varicella-zoster virus antibody fragments to gH neutralize virus while monomeric fragments do not. J Gen Virol. 2001; 82:1959-63. Lantto J, Fletcher J M, Ohlin M. A divalent antibody format is required for neutralization of human cytomegalovirus via antigenic domain 2 on glycoprotein B. J Gen Virol. 2002; 83: 2001-5).

This property of the mAb 4G2 could be common to various antiflavivirus antibodies, as is the case for the chimpanzee antibody 1A5, which recognizes an epitope located also in domain A of the E-protein (Goncalvez A P, Men R, Wemly C, Purcell R H, Lai C J. Chimpanzee Fab fragments and a derived humanized immunoglobulin G1 antibody that efficiently cross-neutralize dengue type 1 and type 2 viruses. J Virol. 2004; 78: 12910-8). In general, the balance between the neutralizing capacity of the mAb and its FAb, depends on the epitope, the identity of the antibody and the stereo-chemical details of the complex. Accordingly, the mAb 4E11 which recognizes an epitope located on domain B, is 50 times more neutralizing that its corresponding FAb (Thullier, P., P. Lafaye, F. Megret, V. Deubel, A. Jouan, and J. C. Mazie. 1999. A recombinant Fab neutralizes dengue virus in vitro. J. Biotechnol. 69:183-190).

Design of Multivalent Neutralizing Molecules

The current invention describes the design and development of molecules capable to bind simultaneously two or three copies of the highly conserved epitope on the virion surface. The virion exposes a total of 180 copies of the conserved epitope described in the present invention. They could be grouped as 90 pairs of epitopes corresponding to E-protein dimers or 60 triplets matching the three copies of E-protein present in the asymmetric unit of the virion. The herein described molecules are capable of divalent or trivalent binding and display an improved binding affinity for the virion and a neutralizing capacity which is various order more potent compared to the neutralizing antibodies recognizing the conserved epitope described in this invention. The described molecules neutralize the four Dengue virus serotypes and other flavivirus and therefore are useful for the prophylactic and/or therapeutic treatment of Dengue and alternatively of other flavivirus.

The sequences of the divalent or trivalent protein molecules of the present invention are described by the following formula:
[S]-[L]-[D] or [S]-[L]-[T],
   where [S] is the sequence of a single chain antibody fragment (scFv), which recognizes the conserved epitope described in this invention, [L] is a linker sequence of size typically between 3-20 amino acids, [D] is the sequence of a protein or its fragment which forms dimers and [T] is a protein or its fragment which forms trimers. The segments [D] and [T] are proteins or protein domains which do not interact with FC receptors capable of mediate immune-enhancement of the viral infection. This way, it is possible to prevent the enhancement of the viral infection of FC receptor bearing cells at sub-neutralizing concentrations of divalent or trivalent molecules of the present invention. Therefore, the described molecules are superior compared to antibodies regarding their incapability to mediate an ADE like effect. Furthermore, these molecules have a larger size compared to the scFvs and hence display a larger half time of life in vivo.

The sequences [D] and [T] correspond to extra-cellular human proteins, preferably from serum. This way it is possible to prevent the induction of an autoantibody response that would appear against intra-cellular and/or foreign proteins.

In general, the domains [D] and [T] could be replaced by multimerization domains capable of forming larger oligomers, if suitable linker sequences are chosen which allow multivalent binding to occur.

The designed multimerization (including dimerization and trimerization) allows increasing the avidity of the fragments and improving their intrinsic capacity of neutralization. Virus binding at multiple points further stabilizes the structure of the mature virion, which interferes with the changes in quaternary structure associated to the membrane fusion process. Moreover, the increase in the molecular size causes a raise in the half time of life in vivo. These recombinant proteins, which include antibody Fv fragments, could become effective therapeutic and/or prophylactic agents for the control of epidemic outbreaks.

The current invention describes a gene which codifies for a chimerical protein named TB4G2. The plasmid pET-TB4G2-LH codifies for the protein TB4G2 fused at the N-terminus to the signal peptide pelB and at the C-terminus to a sequence codifying for 6 histidines (Sequence No. 16).

The chimerical protein TB4G2 contains the following elements from the N- to the C-terminus: (a) the variable domain of the light chain of mAb 4G2 (Sequence No. 25), (b) a flexible spacer sequence (Sequence No. 26), (c) the variable domain of the heavy chain of mAb 4G2 (Sequence No. 27), (d) a flexible spacer sequence of 15 residues (Sequence No. 28), (e) a fragment of human matrilin, which allows the molecule to trimerize in solution (Sequence No. 51).

The chimerical protein TB4G2 corresponds to the topological variant [S]-[L]-[T], where [S] is a scFv fragment of mAb 4G2, [L] is a spacer sequence of 15 residues composed by GLY and SER residues, and [T] is a trimerization domain of human matrilin which forms a helical coiled-coil trimeric structure with the alpha helices aligned in a parallel conformation (Dames S A, Kammerer R A, Wiltscheck R, Engel J, Alexandrescu A T. NMR structure of a parallel homotrimeric coiled coil. Nat Struct Biol. 1998; 5: 687-91).

This matrilin fragment forms covalent trimers stabilized by disulfide bridges formed between cysteins located at the N-terminus of the helix. The signal peptide pelB allows the periplasmic location of the protein TB4G2 and hence its correct folding in vivo, which includes the correct formation of disulfide bridges of the binding domain and the trimerization domain.

According to the models of the complex formed between the virion and the Fv 4G2, the distances measured between the C-terminus of the Fv heavy chains corresponding to Fv fragment bound to the three E-protein monomers of the asymmetric unit are 36, 58 and 70 Å. These three C-terminal atoms are circumscribed in a sphere with a radius of 35 Å, which indicates that the spacer segment [L] must adopt conformations compatible with this distance.

In theory, a segment of 15 residues adopting an extended conformation has a dimension of 52 Å from the N-to the C-terminus. However, such conformation is not necessarily the most stable and in general the structural properties of peptides are determined by their sequences. Peptides rich in GLY and SER are essentially flexible, and are able to adopt multiple conformations in solution. As shown in the example 9, the prediction of peptide conformation using PRELUDE (Rooman M J, Kocher J P, Wodak S J. Prediction of protein backbone conformation based on seven structure assignments. Influence of local interactions. J Mol Biol. 1991; 221:961-79) indicates that the most favorable conformation predicted for the sequence [L] (Sequence No 28) correspond to a distance between the N- and C-terminus of about 35 Å. This finding point out that the design of the chimerical protein TB4G2 is structurally compatible with the capacity of achieving a simultaneous binding to the three E-protein monomers present in the asymmetric unit of the virion.

The chimerical protein TB4G2 was obtained in soluble form in the periplasm of the bacteria E. coli. An easily scalable purification process was developed based on metal chelates chromatography (IMAC), which allowed obtaining pure protein preparations. The purified protein was analyzed by SDS-PAGE electrophoresis. The protein TB4G2 previously treated under reductive condition migrates to a band corresponding to the mass of a monomer and to a trimer when treated under not reductive condition.

Finally, in order to compare the neutralizing capacity of protein TB4G2 with respect to the mAb 4G2 and its fragments FAb and (FAb′)₂, a neutralization test was carried out against the four Dengue virus serotypes in BHK-21 cells. The protein TB4G2 showed similar neutralization titers against the four serotypes, which are two-three orders more potent compared with the antibody and its fragments.

The present invention describes a gene (Sequence No 17), which codifies for a chimerical protein named MA4G2. The chimerical protein MA4G2 (Sequence No 56) contains the following elements from the N-terminus to the C-terminus: (a) the variable domain of the light chain of mAb 4G2 (Sequence No. 25), (b) a flexible spacer sequence (Sequence No. 26), (c) the variable domain of the heavy chain of mAb 4G2 (Sequence No. 27), (d) a flexible spacer sequence of 3 residues (Gly-Gly-Gly), (e) the hinge segment, the CH2 and the CH3 domains of the human IgG1 immunoglobulin molecule. In the CH2 domain of the human IgG1, the protein has been mutated in position ASN297→GLN.

The chimerical protein MA4G2 corresponds to the topological variant [S]-[L]-[D], defined in the present invention, where [S] is a single chain scFv fragment of mAb 4G2, [L] is a three residues spacer segment of sequence GLY-GLY-GLY and [D] is a segment containing the hinge segment, the CH2 and the CH3 domains of the human IgG1 immunoglobulin molecule. The hinge segment mediates the formation of intermolecular disulfide bridges between two identical protein chains, resulting in a stable dimeric structure. The mutation ASN297→GLN in the CH2 domain of the human IgG1 prevents the glycosilation of the protein in Eucariotes and precludes the binding to the FcγR I-III. These receptors mediate the ADE phenomena in vitro. This way, unlikely the mAb 4G2, the designed chimerical protein lacks the risks associated to ADE at sub-neutralizing concentrations. However, the chimerical protein retains the capacity of interacting with the FcRn receptor, a property favorable to achieve longer half time of live in vivo, in a similar manner to the antibody molecules.

The plasmid pET-MA4G2-LH (Sequence No 20) codifies for the protein MA4G2 fused at the N-terminus to the signal peptide pelB (Sequence No 24) and at the C-terminus to a tail of 6 Histidines. The signal peptide pelB allows the localization of the protein MA4G2 in the periplasm, where occurs the correct formation of the intra-molecular disulfide bridges (binding domains, CH2 and CH3) and between the hinge-segments (intermolecular bridges). The histidine tail allows the purification of the protein by metal chelates chromatography.

The 3D model of the complex formed by the protein MA4G2 and the E-protein dimers (example 9), as well as the results of the neutralization tests (example 12) indicate that the chimerical protein MA4G2 is stereo-chemically compatible with a simultaneous binding to the monomers associated as dimers in the structure of the mature virions. This way, bivalency results in a significant increase of the biological activity of the protein.

An essential aspect of the present invention consists in the finding that molecules capable of binding to the herein described highly conserved surface patch of the E-protein, interfere with the biological function of this protein, and such molecules constitute potential candidates for antiviral agents of wide spectrum against flavivirus. As shown in the example 12, fragments of mAb 4G2 including the scFv display a neutralizing activity similar to the whole mAb 4G2, indicating that bivalency is not required for the antiviral activity. These results also show that the antiviral activity of the fragments depends on the interference with the biological activity of the E-protein and this interference is mediated by binding to the described highly conserved area of the protein. Furthermore, the observed antiviral activity is of wide spectrum against flavivirus. Therefore, attractive methods for the identification of antiviral molecules with these properties are those which allow identifying proteins, peptides and drug-like molecules which bind to the described highly conserved surface area. Such methods are those based in blocking the binding to the E-protein of those antibodies which recognize the highly conserved surface area, like the mAb 4G2, its corresponding FAb and (Fab′)2 fragments or the chimerical proteins described in the present invention. Those methods could be based on immune-enzymatic assays, radio-immune assays, assays with fluorescent dyes and these assays allows quantifying the binding of molecules to the E-protein, virions or the herein described chimerical proteins which display the highly conserved surface area.

These assays could be useful to identify potential antiviral molecules effective against a wide spectrum of flavivirus, by means of in vitro screening of libraries of chemical compounds including those generated by methods of combinatorial chemistry.

The identification of candidate molecules could be carried out using computer aided virtual screening methods. These methods are based on computational procedures like the molecular docking of chemical compounds. Using these methods, it is possible to model the binding of chemical compounds to proteins and to quantify the interaction strength or binding energy, which is predicted or calculated from the modeled complex coordinates by means of scoring functions.

Examples of these computational procedures of molecular docking are the programs GOLD (Jones, G. y cols., 1997. Development and validation of a genetic algorithm for flexible docking. J. Mol. Biol. 267, 727-748), DOCK (Kuntz, I. D. y cols., 1982 A geometric approach to macromolecule-ligand interactions. J. Mol. Biol. 161, 269-288) and FLEXX (Olender, R. and Rosenfeld, R., 2001. A fast algorithm for searching for molecules containing a pharmacophore in very large virtual combinatorial libraries. J. Chem. Inf. Comput. Sci. 41, 731-738). These methods allow large virtual libraries of molecules like ZINC database (Irwing, J. J. and Scoichet, B. K., 2005. Zinc—A free Database of commercially available compounds for virtual screening. J. Chem. Inf. Model. 45, 177-182) to be screened and determine which molecules are expected to bind the active site selected on the receptor protein. Regarding the present invention, the binding site is the previously described highly conserved surface area. The crystallographic structures of E-protein available in the PDB database could be used as source for atomic coordinates, or alternatively computational models could be used, which are obtained by means of methods like protein modeling by homology.

### DESCRIPTION OF THE FIGURES

FIG. 1. Graphic representation of the conservation profile corresponding to the surface residues of the flaviviral E-protein. Conservation is represented in a grey scale basis, residues showing more conservation among the flavivirus sequences are darker. The highly conserved surface patch of domain II is encircled. The conservation values were calculated using the program CONSURF, considering a multiple sequence alignment of those flavivirus sequences available in SWISSPROT database. The conservation values were mapped on the protein surface using Pymol.

FIG. 2. Graphic representation of the conservation profile corresponding to the surface residues of the E-protein from Dengue virus. Conservation is represented in a grey scale basis, residues showing more conservation among the flavivirus sequences are darker. The highly conserved surface patch of domain II is encircled. The conservation values were calculated using the program CONSURF, considering a multiple sequence alignment of sequences corresponding to the four Dengue virus serotypes which are available in SWISSPROT database. The conservation values were mapped on the protein surface using Pymol.

FIG. 3. Model of the three dimensional structure of the chimerical protein PMEC1. B is the segment Leu237-Val252 and C is the segment Lys64-Thr120 of the E-glycoprotein of Dengue 2 virus. L is the linker segment consisting of two residues. The 3D model of the protein was obtained using the WHATIF program package and the graphic was made using Pymol.

FIG. 4. Plasmid pET-sPMEC1.

FIG. 5. Plasmid pET-scFv 4G2 LH.

FIG. 6A. Plasmid pET-TB4G2 LH.

FIG. 6B. Plasmid pET-MA4G2 LH.

FIG. 7. Physicochemical characterization of the chimerical protein PMEC1-His6. A: SDS-PAGE electrophoresis of the protein purified by immobilized metal affinity chromatography (lane 1) and reduced and carbamidomethylated (lane 2). B: RP-C4 reversed phase chromatographic analysis of the protein, the arrow indicates the location of the major peak. C: Mass spectra corresponding to the major peak collected by reversed-phase chromatography.

FIG. 8. Summarizing scheme of the results obtained in 13 computational simulation of molecular docking (using the CLUSPRO program) preformed in order to predict the structure of the complex formed by the Fv fragment of the mAb 4G2 and the E-protein from dengue 2. The columns show in a grey scale basis the structural properties of the first 30 solutions (clusters) obtained in each simulation. The solutions are represented by three properties. The first property shows the E-protein domain involved in binding, from lighter to darker gray corresponds to domain II, I and III respectively. Two colors mean simultaneous binding to two domains. L and T means that the epitope involves the linker connecting domains I and III or the fusion peptide respectively (tip of domain II). The second property is represented by three colors, white means binding to the inner surface of the virion, gray is a lateral binding and black means binding to the outer surface of the virion. The third case is the biologically relevant assuming that antibody binding does not depend on major structural changes of the virion structure. The third property correspond to the antibody paratope, gray means that binding involves the antibody CDRs (relevant solutions), white indicates that binding does not involves CDRs (irrelevant solutions). The solutions compatible with the available experimental data are shown using arrows. Their properties correspond to the colors light gray-black-gray. The first two rows located at the top of the graphic indicates the definition of ligand and receptor used in the simulations and includes the PDBfile identifier corresponding to the E-protein crystal structure used in the simulation. The protein-protein docking program (dot or zdock) used in the each simulation is shown below every column.

FIG. 9. Modeling the complex formed between the mature virion from Dengue 2 virus and 180 copies of the FAb 4G2. The model was obtained by docking the previously predicted structure of the FAb4G2-E-protein complex into the structure of the mature virion obtained by cryoelectron micrscopy (1THD). The distances calculated between the C-terminus of the heavy chains of the FAbs bound to three monomers of E-protein found in the asymmetric unit.

FIG. 10. Computer model of the complex formed by the chimerical protein MA4G2 and the E-protein dimer. The figure was obtained using the program Pymol.

FIG. 11. Prediction of conformer stability corresponding to the peptide sequence (GGGS)₃GGG. Energy of conformers is shown as a function of the distance between the N- and the C-terminus. The prediction was performed using the program PRELUDE.

### EXAMPLES

### Example No. 1

### Design of the Chimerical Protein PMEC

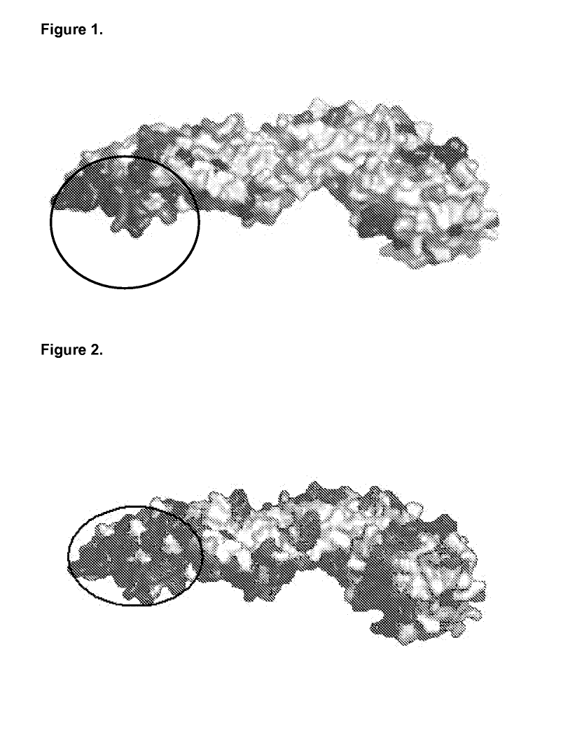

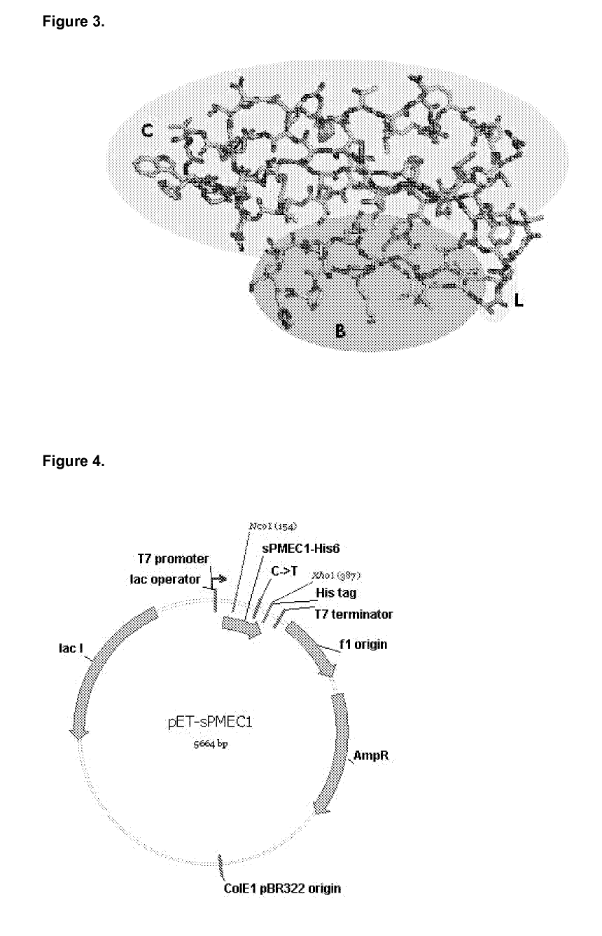

With the aim of identifying conserved patches on the surface of the E-protein, a conservation analysis was carried out using the CUNSURF method (ConSurf: identification of functional regions in proteins by surface-mapping of phylogenetic information. Glaser, F., Pupko, T., Paz, I., Bell, R. E., Bechor-Shental, D., Martz, E. and Ben-Tal, N.; 2003; Bioinformatics 19: 163-164). A highly conserved surface patch is observed on the tip of domain II, which is conserved among the four Dengue virus serotypes and the rest of flavivirus (FIGS. 1 and 2).

The highly conserved surface patch defines a topographic epitope, conformed by residues located close in the three dimensional structure but distant in the sequence of the E-protein. This surface area is comprised on a structural sub-domain located at the extreme of domain II and it is conformed by two lineal segments of E-protein, Leu237-Val252 (segment B) and Lys64-Thr120 (segment C). The table 1 shows the list of residues of the sub-domain, which are located on the outer surface of the virion and hence accessible to the interaction with antibodies. Highly conserved residues define the area or epitope identified by this invention.

The inspection of the structure of domain 11 of E-protein, indicates that the sub-domain presents structurally independent domains like properties. The contact surface to the rest of the protein is 184 Å², which represents only the 12% of the total solvent accessible surface area of the sub-domain. Moreover, this portion of the structure is defined as a structural domain in the CATH database (CATH domain 1svb03, http://www.biochem.ucl.ac.uk/bsm/cath/cath.html).

TABLE 1
Definition of relevant residues of the present invention,
which are located on the extreme of domain II of
E-protein and exposed on the virion surface.

| | No. | | | | |
|---|---|---|---|---|---|
| AA | E DEN-2 | No. PMEC1 | ACC (Å2) | CONS | epitope |
| HIS | 244 | 8 | 40.8 | −1.074/−0.792 | |
| LYS | 246 | 10 | 43 | −0.539/−0.792 | X |
| LYS | 247 | 11 | 41.2 | −0.667/−0.334 | X |
| GLN | 248 | 12 | 4 | −0.702/−0.792 | X |
| ASP | 249 | 13 | 19.5 | 0.366/−0.298 | X |
| VAL | 251 | 15 | 13.8 | 0.726/0.835 | X |
| VAL | 252 | 16 | 11.7 | −0.724/−0.792 | X |
| LYS | 64 | 19 | 26.5 | 0.426/0.271 | X |
| LEU | 65 | 20 | 9.3 | −0.335/−0.383 | X |
| THR | 66 | 21 | 11.7 | 0.210/−0.152 | X |
| ASN* | 67 | 22 | 30.2 | 0.417/−0.792 | X |
| THR | 68 | 23 | 22.3 | 0.952/1.062 | X |
| THR | 69 | 24 | 14.6 | −0.745/−0.792 | X |
| THR | 70 | 25 | 23.7 | −0.781/−0.792 | X |
| GLU | 71 | 26 | 13.3 | 2.146/−2.661 | X |
| SER | 72 | 27 | 12.4 | −0.431/−0.492 | X |
| ARG | 73 | 28 | 21.5 | −0.284/−0.792 | X |
| CYS | 74 | 29 | 12.7 | −1.074/−0.792 | X |
| LEU | 82 | 37 | 6.2 | −0.811/−0.792 | X |
| ASN | 83 | 38 | 39.4 | 4.302/2.327 | X |
| GLU | 84 | 39 | 11.2 | −0.677/−0.792 | X |
| GLU | 85 | 40 | 17 | −0.861/−0.792 | |
| ASP | 87 | 42 | 11.7 | −0.486/−0.792 | |
| ARG | 89 | 44 | 30.8 | 2.051/0.179 | |
| PHE | 90 | 45 | 6.4 | 2.777/4.283 | X |
| VAL | 97 | 52 | 1.7 | −0.766/−0.792 | X |
| ARG | 99 | 54 | 10.5 | −0.898/−0.792 | X |
| GLY | 100 | 55 | 1.3 | −0.796/−0.792 | X |
| TRP | 101 | 56 | 15.4 | −1.074/−0.792 | X |
| GLY | 102 | 57 | 14.8 | −1.074/−0.792 | X |
| ASN | 103 | 58 | 21.7 | −1.074/−0.792 | X |
| GLY | 104 | 59 | 18 | −0.775/−0.792 | X |
| CYS | 105 | 60 | 1.8 | −1.074/−0.792 | X |
| GLY | 106 | 61 | 16.3 | −1.074/−0.792 | X |
| MET | 118 | 73 | 13.6 | −0.877/−0.349 | X |

AA: amino acid,
No. E DEN2: number of the residue in the sequence of E-protein from dengue 2,
No. PMCE1: number of the residue in the sequence of the chimerical protein PMEC1,
ACC: Solvent accessible surface area calculated with WHATIF (Vriend G. WHATIF: a molecular modeling and drug design program. J Mol Graph. 1990; 8: 52-6, 29). Calculations were performed on an atomic model of E-protein, which was obtained by docking independently the 3D structure of the structural domains I, II and III (PDB file 1oan) on the structure of the mature virion (PDB file 1THD).
CONS: Conservation scores calculated with CONSURF, using two sequence alignments, taking into account flavivirus sequences and sequences from the four dengue virus serotypes respectively. Negative values indicate higher conservation and bold highlight the values corresponding to the residues defined as highly conserved,
epitope: Residues making contacts to FAb 4G2 according to the 3D model obtained by molecular docking in the Example 8. Those residues are considered which have at least one atom whose van der waals sphere is separated by less than 3 A from the van der waals sphere of an atom of FAb 4G2,
*ASN22 glycosilated in DEN2 virus.

In order to obtain an independently folded sub-domain, it is necessary in first place to connect the two segments in a unique polypeptide chain. Two possible connections or topologies are possible:
B-L-C y C-L-B
   where L is a linker or spacer segment. The linker needs to be stereo-chemically compatible with the three dimensional structure of the sub-domain and in the best case provide a stabilizing effect on the thermodynamic stability of the chimerical protein. The distance between the alpha carbons of residues Val252 and Lys64 is 6.6 Å, therefore the topology B-L-C could be obtained by the use of linkers of one or more residues. An structural analysis of possible connecting turns on the PDB data base, searching for turns compatible with the structure of the anchor segments (DGINS command in the DGLOOP menu of WHATIF program package) indicates that connections of two residues are more common than connections of one residue. In the case of the topology C-L-B, the distance between the alpha carbons of the residues Thr120 and Leu237 is 11.1 Å, consistent with connections of 3-4 residues or more.

The chimerical protein PMEC1 (sequence 14) of the present invention corresponds to a topology B-L-C, with fragment B and C corresponding to sequences from dengue 2 virus and a two residues Gly-Gly linker sequence.

As B and C segment sequences could be chosen not only the sequences corresponding to DEN2 virus, but also the homolog sequences from other flavivirus, including but not limiting DEN1, DEN3, DEN4, Japanese Encephalitis virus, Tick-born Encephalitis virus, West Nile virus, Murray Valley Encephalitis virus, St Louis Encephalitis virus, LANGAT virus, Yellow Fever virus, Powassan virus (sequences 29-42).

Moreover, the chimerical proteins designed according to the method described above, could be mutated at one or multiple residues, with the aim to increase the thermodynamic stability of the protein or the efficiency of folding process. Those residues described in table 1, which are not accessible to the virion surface and to the interaction with antibodies, could be mutated. The residues susceptible to be mutated are those residues which are buried on the 3D structure of the protein and/or are located in the lateral or inner surface of the 3D/4D structure of the E-protein present in the mature virion.

The mutated protein could be obtained by experimental combinatorial methods like the filamentous phage libraries. The proteins could also be designed using theoretical methods like FOLDX, POPMUSIC and Rosseta.

The sequences 43-50 correspond to analogs of the chimerical protein PMEC1 mutated at multiple positions. Three dimensional models of this proteins show a good packing and quality. Mutations at the exposed surface of the protein are also possible, especially at residues which are not strictly conserved among the Dengue virus serotypes and other flavivirus, with the condition that these mutations must not affect the interaction with protective and neutralizing antibodies recognizing the conserved sub-domain of E-protein.

### Example No. 2

### Construction of Plasmid pET-sPMEC1

In order to obtain a recombinant gene coding for the protein PMEC1 (Sequence No. 1), the gene coding for protein E from the DEN2 virus (Sequence No. 2), strain 1409, genotype Jamaica, present on plasmid p30-VD2 (Deubel V., Kinney R. M., Trent D. W.; “Nucleotide sequence and deduced amino acid sequence of the structural proteins of dengue type 2 virus, Jamaica genotype”, Virology 155(2):365-377, 1986) was used. This gene codes for the protein shown in Sequence No. 3. Using the method of Agarwal et al. (Agarwal K L, Büchi H, Caruthers M H, Gupta N, Khorana H G, Kumas A, Ohtsuka E, Rajbhandary U L, van de Sande J H, Sgaramella V, Weber H, Yamada T, Total synthesis of the Gene for an alanine transfer ribonucleic acid from yeast, 1970, Nature 227, 27-34), and starting from oligonucleotides synthesized on solid phase by phosphoramidite chemistry (Beaucage S L, Caruthers M H, Deoxynucleoside phosphoramidites—A new class of key intermediates for deoxypolynucleotide synthesis., Tetrahedron Letters, 1981, 22, 1859), a double stranded DNA molecule coding for the PMEC1 protein was obtained (Sequence No. 4). The sequence of this DNA molecule has the following elements: 1) A recognition site for the Nco I restriction enzyme, containing the start codon coding for the aminoacid methionine (M), followed by a codon coding for the aminoacid Alanine (A) (Sequence No. 5); 2) A fragment corresponding to the sequence, from position 709 to position 756, of the gene for protein E of virus Dengue 2 strain Jamaica 1409 (Sequence No. 6), coding for the peptide sequence shown in Sequence No. 7, that in turn corresponds to positions 237 to 252 of Sequence No. 3; 3) A linker segment coding for two successive Glycines (Sequence No. 8); 4) A fragment corresponding to the sequence spanned by positions 190 to 360 of Sequence No. 2, which codes for the peptide sequence shown in Sequence No. 9 (which corresponds to positions 64-120 of Sequence No. 3), where a silent mutation has been introduced that eliminates the Nco I restriction site present in positions 284-289 of said sequence (Sequence No. 10); and 5) A recognition site for the Xho I restriction enzyme, containing two codons that code for the aminoacids Leucine (L) and Glutamic acid (E), respectively (Sequence No. 11). This synthetic molecule was digested with the Nco I and Xho I restriction enzymes (Promega Benelux b.v., The Netherlands) in the conditions specified by the manufacturer, and ligated using T4 DNA ligase (Promega Benelux, b.v., The Netherlands), in the conditions specified by the manufacturer, to plasmid pET22b (Novagen, Inc., USA) previously digested identically. The reaction was transformed into the Escherichia coli strain XL-1Blue (Bullock W O, Fernandez J M, Short J M. XL-1Blue: A high efficiency plasmid transforming recA Escherichia coli K12 strain with beta-galactosidase selection. Biotechniques 1987;5:376-8) according to Sambrook et al. (Sambrook J, Fritsch E F, Maniatis T. Molecular cloning: A laboratory manual. New York, USA: Cold Spring Harbor Laboratory Press; 1989), and the plasmids present in the surviving colonies in selective medium were screened by restriction analysis. One of the resulting recombinant plasmids was denominated pET-sPMEC1 (FIG. 4), and its sequence (Sequence No. 12) was verified through automatic Sanger sequencing.

The plasmid pET-sPMEC1 codes for the protein PMEC1 fused, on its N-terminal end, to the pelB leader peptide and, on its C-terminal end, to a sequence coding for 6 histidines (Sequence No. 13). This arrangement allows, on one hand, the processing of this protein in the host through cleavage of the leader peptide and secretion to the E. coli periplasm, where the prevailing oxidizing conditions facilitate correct folding and formation of the disulphide bridges of PMEC1, and also allows, on the other hand, easy purification of this protein through immobilized metal affinity chromatography (IMAC) (Sulkowski, E. (1985) Purification of proteins by IMAC. Trends Biotechnol. 3, 1-7). The final sequence of the protein, denominated PMEC1-His6, after processing and secretion to the periplasm, is shown in Sequence No.14.

### Example No. 3

### Expression and Purification of PMEC1-His6

Plasmid pET-sPMEC1 was transformed (Sambrook J, Fritsch E F, Maniatis T. Molecular cloning: A laboratory manual. New York, USA: Cold Spring Harbor Laboratory Press; 1989) into the E. coli strain BL21 (DE3) (Studier, F. W and B. A. Moffatt. “Use of bacteriophage T7 RNA polymerase to direct selective high-level expression of cloned genes.” J. Mol. Biol. 189.1 (1986): 113-30), and a 50 mL culture of Luria-Bertani medium supplemented with ampicillin at 50 μg/mL (LBA) was inoculated with a single, isolated colony and grown for 12 hours at 30° C. at 350 r.p.m. With this culture, 1 L of LBA medium was inoculated to a starting optical density at 620 nm (OD620) of 0.05, which was then grown for 8 h at 28° C. until late exponential phase. This culture was then induced by the addition of isopropylthiogalactoside (IPTG), and grown in the same conditions for an additional period of 5 hours.

The culture obtained as described above was centrifuged at 5000×g for 30 min. at 4° C. and the periplasmic fraction was extracted from the resulting biomass using the method of Ausubel et al. (Ausubel, F. M., Brent, R., Kingston, R. E., Moore, D. D., Seidman, J. G., Smith, J. A. and Struhl, K (1989) in Current Protocols in Molecular Biology. John Wiley & Sons, New York). This fraction was dialyzed against 50 mM phosphate buffer pH 7/20 mM imidazole using a membrane with a 1000 Da cut-off, and protein PMEC1-His6 was obtained from the dialyzate by immobilized metal affinity chromatography (Sulkowski, E. 1985, Purification of proteins by IMAC. Trends Biotechnol. 3, 1-7), using Ni-NTA agarose (Qiagen Benelux B. V., The Netherlands) following the instructions of the manufacturer.

### Example No. 4

### Physical and Chemical Characterization of the Chimeric Protein PMEC1-His6

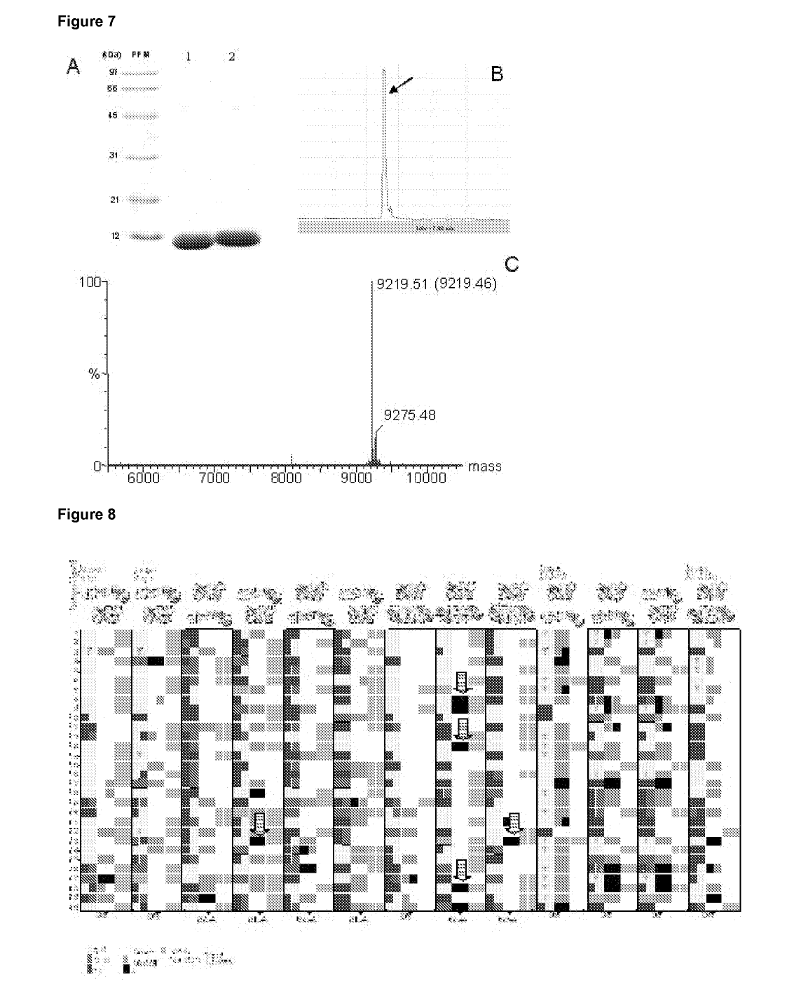

The preparation of PMEC1-His6 purified by IMAC shows a major band on SDS-PAGE (FIG. 7A) that migrates at an apparent mass corresponding to that expected for this protein (approximately 9500 Da), evidencing the high degree of purity of the preparation. The same figure shows that the band corresponding to reduced and carbamidomethylated PMEC1-His6 (lane 2, FIG. 7A) has a slightly reduced electrophoretic migration when compared to the non-reduced sample (Lane 1, FIG. 7A). This behavior indicates that the protein is properly folded, with the cysteines involved in intramolecular disulphide bridges.

A 80 μg aliquot of PMEC1-His6 was analyzed in a C4 4.6×250 mm (J. T. Baker, USA) reversed-phase HPLC column. The chromatographic run was carried out at 37° C., using a high pressure chromatographic system fitted with two pumps and a controller. The elution of the protein was achieved by a 10 to 60% (v/v) linear acetonitrile gradient in 0.1% (v/v) trifluoroacetic acid, at a flow of 0.8 mL/min. and with the detection filter set at 226 nm. The chromatogram yielded a single peak, confirming the high homogeneity of the preparation (FIG. 7B).

The peak from the RP-HPLC was analyzed by mass spectrometry with the goal of measuring the molecular mass of the protein with a higher accuracy and verifying the oxidation status of the disulphide bridges. The spectra were acquired on a hybrid mass spectrometer with octagonal geometry QTOF-2TM (Micromass, UK), fitted with a Z-spray electronebulization ionization source. The acquired spectra were processed using the MassLynx version 3.5 (Micromass, UK) software application. The mass spectrum of the major species of the PMEC1-His6 preparation has a molecular mass of 9219.51 Da (FIG. 7C), and this value is only 0.05 Da off the expected value according to the sequence of the gene. This analysis confirmed that the cysteines residues on the molecule are engaged in disulphide bridges, and discarded the presence of undesired post-translational modifications such as N- or C-terminal degradation or modification of susceptible aminoacids.

### Example No. 5

### Antigenic Characterization of PMEC1

Purified PMEC1 protein was characterized by dot blotting using monoclonal and polyclonal murine antibodies as well as dengue reactive human sera (table 2 and 3). As negative control was employed the recombinant protein DIIIe2 consisting on the domain III of the E protein of Den-2 virus (genotype Jamaica) fused to a hexa-histidine tag. Different from PMEC1, DIIIe2 corresponds to a region of higher sequence variability on the E protein. Recombinant domain III is strongly recognized by anti-Den hyperimmune ascitic fluids (HIAF) exhibiting a marked specificity for the homologous serotype and losing most of the reactivity for the reduction of the disulfide bond of this domain. This serotype specificity in the reactivity of antibodies toward domain III has also been found in human antibody response. Mab 3H5 was also included as a control in the assay. Different from Mab 4G2, 3H5 recognizes a serotype specific epitope within domain III of Den-2. Reactivity of these two Mabs is affected by the treatment with a reducing agent (Roehrig J T, Volpe K E, Squires J, Hunt A R, Davis B S, Chang G J. Contribution of disulfide bridging to epitope expression of the dengue type 2 virus envelope glycoprotein. J Virol. 2004; 78: 2648-52).

TABLE 2
Reactivity of monoclonal and polyclonal antibodies
toward PMEC1 protein in dot blotting

| | | | PMCE1- | | |
|---|---|---|---|---|---|
| Abs** | Specificity*** | PMEC1* | RC* | DIIIe2 | DIIIe2_RC |
| HIAF | DEN-1 | +++ | − | + | − |
| HIAF | DEN-2 | +++ | − | +++ | + |
| HIAF | DEN-3 | +++ | − | + | − |
| HIAF | DEN-4 | +++ | − | + | − |
| HIAF | TBE | ++ | − | − | − |
| HIAF | YFV | ++ | − | − | − |
| HIAF | SLV | +++ | − | − | − |
| Mab | GF | +++ | − | − | − |
| 4G2 | | | | | |
| Mab | DEN-2 | − | − | +++ | − |
| 3H5 | | | | | |

*Ten micrograms of purified PMEC1 and DIIIe2 proteins were loaded to nitrocellulose membrane. RC: reduced and carbamidomethylated protein. Signal intensity was evaluated in a scale of + to +++.
**HIAFs were used at 1:100 dilution. 3H5 and 4G2 Mabs were employed at 10 μg/mL.
***TBE: Tick borne encephalitis virus, YFV: Yelow fever virus, SLV, Saint Louis virus, GF: cross-reactive to flavivirus serogroup.

PMEC1 was recognized by HIAF obtained against the four serotypes of Den as well as for the Mab 4G2. Among the rest of the HIAF obtained against different flaviviruses that were evaluated, anti-SLE exhibited the highest reactivity toward PMEC1 with similar signal intensity as obtained for anti-Den HIAF. Anti-TBE and anti-YF HIAF also recognized PMEC1 even though with lower intensity. Reactivity of the different HIAF was highly dependent on the presence of the disulfide bonds of indicating that the protein is correctly folded in a similar conformation as the native structure of E protein on the virus.

PMCE1 protein was also characterized in dot blotting through the reactivity with human sera from persons that had been infected with Den on different epidemiological situations. In the assay were included sera from convalescents of primary infection with the four virus serotypes (i.e. Den1, Den2, Den3 and Den4). Sera from individuals that had suffered secondary infection with Den2 or Den3 were also used. Human antibodies were employed as pools of sera from three individuals infected in the same epidemic and that experimented similar clinical symptoms and with similar serology results. Each serum was evaluated for the presence of IgM antibodies against the viral antigens and PMEC1 protein.

TABLE 3
Reactivity of human antibodies toward PMEC1 protein in dot blotting.

| | PI* | | | | SI** | | | |
|---|---|---|---|---|---|---|---|---|
| | DEN-1 | DEN-2 | DEN-3 | DEN-4 | DEN-2 | DEN-3 | Mab 4G2 | Mab 3H5 |
| PMEC1*** | ++ | ++ | ++ | ++ | +++ | +++ | +++ | − |
| PMCE1-RC | − | − | − | − | − | − | − | − |
| DIIIe2 | − | ++ | − | − | ++ | ++ | − | +++ |
| DIIIe2-RC | − | − | − | − | − | − | − | − |
| DEN Ag | +++ | +++ | +++ | +++ | +++ | +++ | +++ | ++ |
| Neg ctrl | − | − | − | − | − | − | − | − |

*Pool of sera from convalescents of primary infection for (PI) for DEN-1, DEN-2, DEN-3 and DEN-4.
**Pool of sera from convalescents of secondary infection for (SI) for DEN-2 and DEN-3.
***Ten micrograms of purified PMEC1 and DIIIe2 proteins were loaded to nitrocellulose membrane. RC: reduced and carbamidomethylated protein. Signal intensity was evaluated in a scale of + to +++.
DEN Ag: Pool of viral antigens of the four serotypes. Viral antigens were obtained from the supernatant of infected Vero cells.
Neg Ctrl: Control preparation consisting on the supernatant of non-infected Vero cells.
Human sera were used at 1:400 dilution. 3H5 and 4G2 Mabs were employed at 10 μg/mL.

Human sera from individuals infected with the different virus serotypes recognized PMEC1 with similar intensity. Strongest signals were obtained with sera from individuals that suffered secondary infection which corresponded with the higher anti-viral titers by ELISA as well.

### Example No. 6

### Characterization of Antibody Response Obtained by Immunization with PMEC1 Protein

A group of 80 Balb/c mice were injected by intraperitoneal (i.p) route with 20 μg of purified PMEC1 emulsified with Freund's adjuvant. Ten mice were bled after the fourth dose and the sera were collected for further serological analysis. The anti-Den antibody titers measured by ELISA were similarly high for the four serotypes of the virus (Table 4). In parallel, the functionality of the Abs elicited was measured by inhibition of hemaglutination (IHA) and plaque reduction neutralization (PRNT) tests. In the IHA assay anti-PMEC1 antibodies yielded positive titers against the four Den serotypes (Table 5). Also neutralization titers of 1/1280 were obtained against the four viruses (Table 6).

TABLE 4
Anti-Den antibodies titer of sera obtained
by immunization with PMEC1 protein.

| | Antibody titer determined by ELISA* | | | |
|---|---|---|---|---|
| mouse | anti-DEN-1 | anti-DEN-2 | anti-DEN-3 | anti-DEN-4 |
| 1 | 1:128 000 | >1:128 000 | 1:64000 | 1:128 000 |
| 2 | >1:128 000 | >1:128 000 | >1:128 000 | >1:128 000 |
| 3 | 1:64000 | >1:128 000 | 1:128 000 | 1:128 000 |
| 4 | >1:128 000 | >1:128 000 | >1:128 000 | >1:128 000 |
| 5 | 1:128 000 | 1:128 000 | 1:128 000 | 1:32000 |
| 6 | 1:64000 | 1:64000 | >1:128 000 | 1:64000 |
| 7 | >1:128 000 | >1:128 000 | >1:128 000 | >1:128 000 |
| 8 | 1:128 000 | 1:64000 | 1:128 000 | 1:128 000 |
| 9 | >1:128 000 | >1:128 000 | >1:64 000 | >1:128 000 |
| 10 | >1:128 000 | >1:128 000 | >1:64 000 | >1:128 000 |

*Antibody titers were determined by end-point dilution. Each serum was evaluated in parallel with a viral antigen preparation obtained from suckling mice brain infected with each virus serotype as described (Clarke, D. M., Casals, J. Techniques for hemaglutination and hemaglutination-inhibition with arthropode-borne viruses. American Journal of Tropical Medicine and Hygiene 1958. 7: 561-573). A similar preparation obtained from brain of non-inoculated mice was used as negative control.

TABLE 5
IHA titer of antibodies generated
by immunization with PMEC1 protein.

| | Titer of IHA* | | | |
|---|---|---|---|---|
| Mouse | anti-DEN-1 | anti-DEN-2 | anti-DEN-3 | anti-DEN-4 |
| 1 | 1:640 | 1:320 | 1:640 | 1:640 |
| 2 | 1:640 | 1:640 | 1:640 | 1:320 |
| 3 | 1:320 | 1:320 | 1:320 | 1:320 |
| 4 | 1:10 | 1:5 | 1:10 | 1:10 |
| 5 | 1:640 | 1:640 | 1:640 | 1:640 |
| 6 | 1:640 | 1:320 | 1:640 | 1:640 |
| 7 | 1:640 | 1:640 | 1:640 | 1:640 |
| 8 | 1:1280 | 1:640 | 1:1280 | 1:1280 |
| 9 | 1:320 | 1:320 | 1:320 | 1:640 |
| 10 | 1:10 | 1:5 | 1:10 | 1:10 |

*IHA titers were defined as the maximal dilution inhibiting goose erythrocytes hemaglutination caused by 8 hemaglutinating viral units.

TABLE 6
Viral neutralization assay using sera from
animals immunized with PMEC1 protein

| | Viral neutralization titer* | | | |
|---|---|---|---|---|
| Mouse | anti-DEN-1 | anti-DEN-2 | anti-DEN-3 | anti-DEN-4 |
| 1 | 1:1280 | 1:1280 | 1:1280 | 1:1280 |
| 2 | 1:1280 | 1:1280 | 1:1280 | 1:640 |
| 3 | 1:640 | 1:160 | 1:320 | 1:320 |
| 4 | 1:80 | <1:40 | <1:40 | <1:40 |
| 5 | 1:1280 | 1:1280 | 1:1280 | 1:1280 |
| 6 | 1:640 | 1:1280 | 1:1280 | 1:1280 |
| 7 | 1:320 | 1:640 | 1:640 | 1:640 |
| 8 | 1:1280 | 1:1280 | 1:1280 | 1:1280 |
| 9 | 1:1280 | 1:320 | 1:1280 | 1:640 |
| 10 | <1:40 | 1:320 | 1:320 | 1:320 |

*Neutralizing titers were defined as the dilution yielding 50% reduction of viral plaques in BHK-21 cells.

### Example No. 7

### Protection Assay

Animals immunized with PMEC1 that remained after bleeding were divided in groups and used to perform the challenge study. Four groups of 15 animals were inoculated by i.c injection with 100 LD50 of a live neuroadapted strain of one of the four serotypes of the virus and observed for 21 days. A fifth group of 10 animals did not received the viral challenge. Positive controls consisted on groups of 15 animals immunized and subsequently challenge with the homologous serotype of the virus (i.e. Den1, Den2, Den3 and Den4). Another four groups of 15 mice each were employed as negative controls of the experiment; these animals received PBS as immunogen and were challenge with the different viral serotypes. Groups of animals immunized with PMEC1 exhibited between 80% to 90% of animal survival, while mice immunized with PBS develop symptoms of encephalitis between days 7-11 after viral inoculation and died before day 21 (Table 7). One hundred percent of the animals from the virus-immunized groups were protected.

TABLE 7
Survival percentage in mice immunized with the protein
variants to the challenge with lethal Den virus.

| | | Survival |
|---|---|---|
| Immunogen | Virus serotype | percentage* |
| PBS | DEN-1 | 0 |
| PBS | DEN-2 | 0 |
| PBS | DEN-3 | 0 |
| PBS | DEN-4 | 0 |
| DEN-1 | DEN-1 | 100 |
| DEN-2 | DEN-2 | 100 |
| DEN-3 | DEN-3 | 100 |
| DEN-4 | DEN-4 | 100 |
| PMEC1 | DEN-1 | 86 |
| PMEC1 | DEN-2 | 80 |
| PMEC1 | DEN-3 | 90 |
| PMEC1 | DEN-4 | 86 |

*Calculated with the following expression: (# of surviving mice)/(# of total mice) Surviving mice data was collected 21 days after challenge.

### Example 8

### Structure Prediction of the Complex Formed by mAb 4G2 and the E-Protein

In order to model the structure of the antigen-antibody complex, a molecular docking study was performed using the crystallographic structure of the FAb fragment of the mAb 4G2 (1uyw) and two crystallographic structure of the envelope E-protein from dengue 2 virus (PDB files 1oan and 1oam). The CLUSPRO method was used for predictions (http://nrc.bu.edu/cluster/, S. R. Comeau, D. W. Gatchell, S. Vajda, C. J. Camacho. ClusPro: an automated docking and discrimination method for the prediction of protein complexes. (2004) Bioinformatics, 20, 45-50), including two different programs for generation of the structures of potential complexes: DOT and ZDOCK (Mandell J G, Roberts V A, Pique M E, Kotlovyi V, Mitchell J C, Nelson E, Tsigelny I, Ten Eyck L F. (2001) Protein docking using continuum electrostatics and geometric fit. Protein Eng 14:105-13. Chen R, Li L, Weng Z (2003) ZDOCK: An Initial-stage Protein-Docking Algorithm. Proteins 52:80-87).

In total, 13 molecular docking simulations were carried out changing the following parameters: the docking program (DOT or ZDOCK), the definition of the ligand and the receptor (Fv fragment or E-protein), the crystallographic structure of the E-protein (1oan or 1oam), the quaternary structure of the E-protein (monomer or dimer), the use of constrains to filtrate solutions which involve the binding site of the Fv fragment or the N-terminal segment (residues 1-120) of the E-protein (Attract option in DOT). The FIG. 7 shows a scheme summarizing the results of the simulations. Those complexes were considered as potential solutions, which predict an epitope localized in the domain II of the E-protein, this epitope is accessible to the interaction with antibodies according to the structure of the virion and the paratope is the hyper-variable region of the antibody. The location of the epitope A1 (epitope recognized by mAb 4G2) in domain II is supported by experimental data and it has also been determined that related antibodies directed against the same epitope, recognize a proteolytic fragment consisting in amino acids 1-120 of the E-protein (Roehrig, J. T., Bolin, R. A. and Kelly, R. G. Monoclonal Antibody Mapping of the Envelope Glycoprotein of the Dengue 2 Virus, Jamaica. 1998, Virology 246: 317-328).

Six potential solutions were obtained, which were structurally very similar. The table 8 shows the values corresponding to the interface parameter of the predicted E-protein-Fv complex, the values calculated for the predicted complex are similar to those calculated for protein-antibody complexes, whose crystallographic structure has been previously determined experimentally (table 9).

The surface patch of the E-protein contacting the antibody, involves 4 segments of the protein sequence. This finding is consistent with the topographic nature of the epitope, whose recognition depends on the correct folding of the protein, and is susceptible to reduction of the disulfide bridges. The structural epitope defined by the three-dimensional model contains region highly conserved in flavivirus, which is consistent with the wide cross-reactivity of this antibody and with the recognition of the chimerical protein PMEC1 shown the example 5. The model also suggests that the neutralization mechanism of this antibody involves the interfering of E-protein binding to membranes and/or the trimerization associated to the fusion process.

Moreover, the epitope recognized by the antibody coincides with the region involved in the interaction between the E-protein and pre-M, as inferred from the electronic density corresponding to preM in the cryo-electron microscopy studies of the immature virions. The evolutionary pressure related to the conservation of the interaction surface could explain the high conservation of this epitope on the E-protein. Furthermore, the appearance of escape mutants in this surface patch is less probable, because such mutations should be compensated by stabilizing simultaneous mutations on the surface of pre-M. In fact, escape mutants obtained against this antibody are located in the hinge region between domains I and II, and the mutant viruses show a highly attenuated and defects in its capacity to fusion membranes. This constitutes a favorable property the PMEC chimerical proteins of the present invention as recombinant vaccine candidates against flavivirus.

Thereafter, we modeled the interaction between the FAb 4G2 and the E-protein, in the context of the structure of the mature virions. With this aim, we docked the previously modeled complex into the cryo-electron microscopy structure of the mature virions. In order to obtain the model we used: 1) the PDB file 1THD corresponding to the structure of the virion obtained by cryo-electron microscopy , 2) the coordinates of the complex formed by the FAb 4G2 and the monomer of the E-protein, which was previously modeled by molecular docking, 3) the icosahedrical symmetry operations corresponding to the file 1THD were applied to the complex previously modeled by molecular docking.

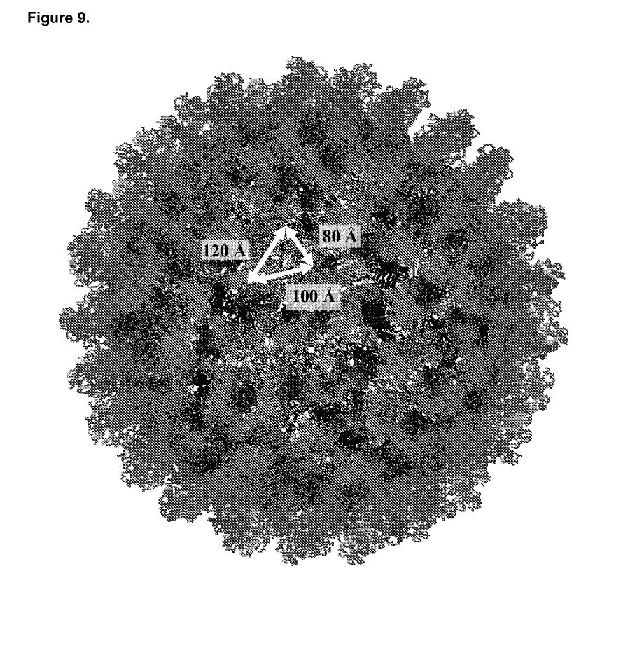

This way, a model was obtained in which all copies of the epitope recognized by the mAb 4G2 (180) present on the virion, are occupied by FAbs (FIG. 9).

TABLE 8
Interface properties of the model corresponding to the
complex formed by the mAb 4G2 and the E-protein.

| Interface parameters* | Value for Fv | Value for E |
|---|---|---|
| Interface surface accessibility | 1070.14 | 1034.10 |
| % Interface surface accessibility | 10.60 | 5.20 |
| Planarity | 2.46 | 2.50 |
| Length y Width | 32.62 & 24.75 | 39.74 & 26.44 |
| Ratio Length/Width | 0.67 | 0.55 |
| Interface segments | 7 | 4 |
| % Interface polar atoms | 51.78 | 46.49 |
| % Interface apolar atoms | 48.20 | 53.50 |
| Secondary structure | Beta | Beta |
| Hydrogen bonds | 16 | 16 |
| Salt bridge | 0 | 0 |
| Disulfide bridge | 0 | 0 |
| Separation Volume | 4618.96 | 4618.96 |
| Index of separation volume | 2.20 | 2.20 |

*protein-protein interface parameters were calculated using the following web server http://www.biochem.ucl.ac.uk/bsm/PP/server/

An inspection of the distance between the C-terminal residues of the heavy chains of the FAb fragments indicates that antibody bivalent binding is not possible without major changes in the structure of the virion. This observation is consistent with the results obtained in the example 12, showing that equimolar amounts of FAb and mAb display very similar neutralizing titers. This finding contrast with an increase of 2-3 orders of magnitude expected for a bivalent binding mode.

TABLE 9
Characteristic interface properties of protein-antibody complexes*.

| Interface parameters | | Protein-antibody |
|---|---|---|
| No of examples | | 6 |
| Interface ASA (Å²) | Media | 777 |
| | sd | 135 |
| Planarity | Media | 2.2 |
| | sd | 0.4 |
| Circularity | Media | 0.8 |
| | sd | 0.1 |
| Segmentation | Media | 4 |
| | sd | 1.83 |
| Hydrogen Bonds per | Media | 1.1 |
| 100(Å²) | sd | 0.5 |
| Separation index | Media | 3.0 |
| | sd | 0.8 |

*Jones, S. and Thornton, J. M. (1996). Principles of Protein-Protein Interactions Derived From Structural Studies PNAS. Vol. 93 p. 13-20. http://www.biochem.ucl.ac.uk/bsm/PP/server/

### Example 9

### Design of the Chimerical Proteins MA4G2 (Bivalent) and TB4G2 (Trivalent)

In this example we show the design of chimerical proteins related to the mAb 4G2 binding site, which can bind simultaneously two or three copies of E-protein monomers displayed in the mature virion. The modeling studies of the example 8 indicates that the distances separating the C-terminal residues of the heavy chains of FAbs associated to E-protein monomers located in the asymmetric unit of the virion are 80, 100 and 120 Å respectively. These values are too large to allow antibody bivalent binding to the virion (FIG. 9). The distances calculated between C-terminal residues corresponding to Fabs bound to E-protein monomer from different asymmetric units are still larger. However, the distances calculated between the C-terminal residues of the heavy chains of Fv fragments bound to the asymmetric unit are 36, 58 and 70 Å respectively. These three atoms are circumscribed in a circle of 35 Å in radius, indicating that trivalent binding is possible by the fusion to trimerization domains through linker segments of 10-15 residues.

Similarly, the corresponding distance between the C-terminus of the heavy chains of Fv fragments bound to E-protein dimers is 36 Å, indicating that bivalent binding is possible by the fusion to dimerization domains with small linker segments of 5-10 residues.

Design of a Miniantibody Type Molecule (Bivalent Binding)

As an example of a bivalent binding molecule we designed the chimerical protein MA4G2. Its sequence contains from the N- to the C-terminus the following segments:
1—scFv, single chain Fv fragment of the mAb 4G2 of the type VL-linker-VH (Sequence No. 25, 26 and 27), VL is the variable region of the light chain of mAb 4G2 and VH is the variable region of the heavy chain of the same antibody.
2—GGG, three glycine residues linker segment
3—Hinge-CH2-CH3, corresponds to the sequence of the hinge segment and the constant domains 2 and 3 of the human IgG1 molecule and the glycosilation site N297 have been mutated to Glycine (Sequence No. 52)

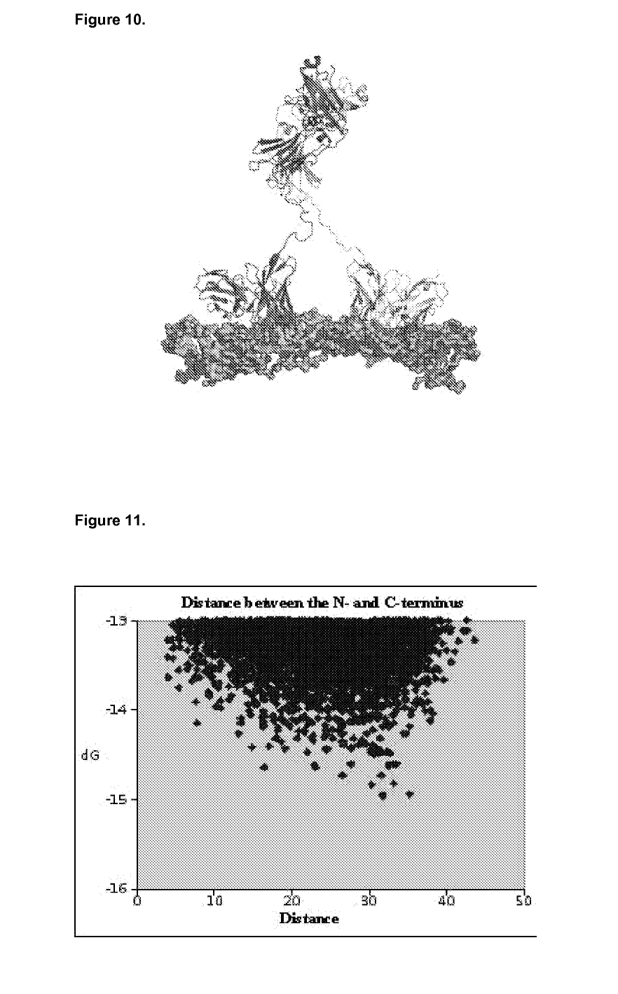

The protein MA4G2 can be expressed in eucariotes and procariotes, and it associates as dimers due to the formation of intermolecular disulfide bridges between the cystein residues located the hinge region, this way displaying a human FC domain at the C-terminal part of the molecule. The hinge region displays adequate spacing and flexibility and therefore a three residue linker (GGG) is enough as connector between the scFv domain and the hinge-FC segment. The FIG. 10 shows a model of the 3D structure of the complex formed by the chimerical protein MA4G2 and an E-protein dimer, indicating the feasibility of bivalent binding to the virion.

The presence of the mutation at the glycosilation site allows obtaining non-glycosilated FC bearing molecules in Eucariotes. The non-glycosilated FC domains do not bind to the receptors FcγRI-III, which are able to mediate infection immune-enhancement in vitro (Lund, J., Takahashi, N., Pound, J. D., Goodall, M., and Jefferis, R. 1996, J. Immunol. 157, 4963-4969. Lund, J., Takahashi, N., Pound, J. D., Goodall, M., Nakagawa, H., and Jefferis, R. 1995, FASEB. J. 9, 115-119). This way, unlike the original antibody molecule, the designed protein lack any risk of mediate ADE at sub-neutralizing concentrations. Furthermore, the chimerical protein retains the FcRn receptor binding properties, which is desired to display long half time of life in vivo, similar to the natural antibodies.

Chimerical Trivalent Protein TB4G2

As an example of a trivalent binding molecule, we designed the chimerical protein TB4G2, whose sequence is described as following structure:
scFv-Linker-T
   where,
1—scFv, single chain Fv fragment of the mAb 4G2 of the type VL-linker-VH (Sequence No. 25, 26 and 27), VL is the variable region of the light chain of mAb 4G2 and VH is the variable region of the heavy chain of the same antibody
2—Linker, is a linker segment of sequence (GGGS)₃GGG (Sequence No. 28)
3—T is a helical trimerization domain human matrilin (Sequence No. 51)

The trimerization domain of matrilin is an alpha helix which trimerizes as a parallel coiled-coil structure. The trimer includes six intermolecular disulfide bridges formed by two cystein residues located close to the N-terminus of each monomer. This trimeric helicoidal structure is highly stable dG=7 kcal/mol at 50° C. (Wiltscheck R, Kammerer R A, Dames S A, Schulthess T, Blommers M J, Engel J, Alexandrescu A T. Heteronuclear NMR assignments and secondary structure of the coiled coil trimerization domain from cartilage matrix protein in oxidized and reduced forms. Protein Sci. 1997; 6: 1734-45). The disulfide bridges ensure the covalent linked trimeric quaternary structure even at very low concentrations, which compares favorably with trimers based in non-covalent interactions only.

The linker segment is composed by the amino acids Gly and Ser and it is very flexible. Amino acid sequences of similar composition have been used very often as linker sequences in protein engineering. Although a segment of 10 residues can provide an spacing of 35 Å necessary for trivalent binding to the virion, it is only true if the segment adopt a fully extended conformation. In solution, the linker segment can display multiple conformations in thermodynamic equilibrium and adopting a unique extended conformation would imply a significant entropic energetic lost. In order to explore the structural properties of the linker segment, we performed a structure prediction of the 15 residue (GGGS)₃GGG sequence using the program prelude. This method is based on statistical potentials describing local interactions and it has been previously used for peptide structure prediction. The FIG. 11 shows a plot of the energy values vs the distance between the N- and C-terminus for the predicted more favorable conformations. The energy minimum corresponds to dimensions of 35 Å and the most extended conformations (more than 40 Å) are very unfavorable. Therefore, the computations indicate that the sequence chosen as linker segment is adequate for the design of trivalent binding molecules.

### Example No. 10

### Obtention of Plasmids Coding for a Single-Chain Antibody Fragment (scFv 4G2), a Trivalent Molecule (TB4G2), and a Single-Chain Miniantibody (MA4G2) with the Variable Regions from Antibody 4G2

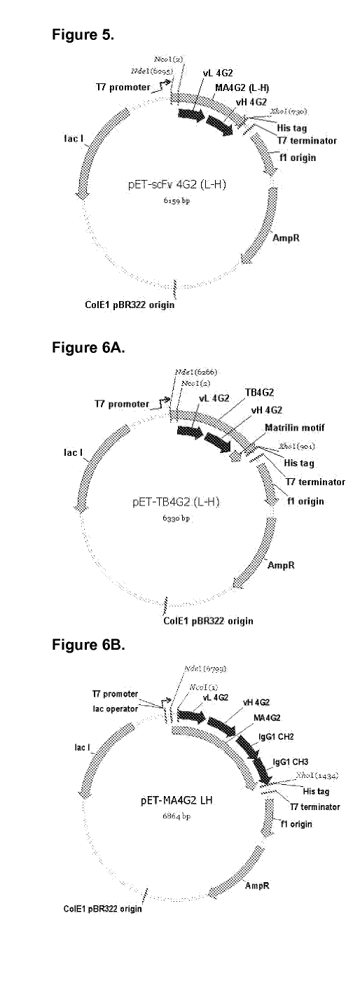

In order to obtain a single chain antibody fragment, a multimeric protein, and a single chain miniantibody (MA4G2) with the variable regions from monoclonal antibody 4G2, the method of Agarwal et al. (Agarwal K L, Büchi H, Caruthers M H, Gupta N, Khorana H G, Kumas A, Ohtsuka E, Rajbhandary U L, van de Sande J H, Sgaramella V, Weber H, Yamada T, Total synthesis of the Gene for an alanine transfer nbonucleic acid from yeast, (1970), Nature 227, 27-34) was used to synthesize, starting from oligonucleotides synthesized on solid phase through phosphoramidite chemistry (Beaucage S L, Caruthers M H, Deoxynucleoside phosphoramidites—A new class of key intermediates for deoxypolynucleotide synthesis., Tetrahedron Letters, (1981), 22, 1859), double stranded DNA molecules (Sequence No. 15, Sequence No. 16 and Sequence No. 17), each of which was digested with the restriction enzymes Nco I and Xho I (Promega Benelux b.v., The Netherlands) under the conditions specified by the manufacturer. Each digested molecule was then ligated using T4 DNA ligase (Promega Benelux, b.v., The Netherlands), under the conditions specified by the manufacturer, to plasmid pET22b (Novagen, Inc., USA), previously digested with the same enzymes. The ligations were transformed into the E. coli strain XL-1 Blue (Bullock W O, Fernandez J M, Short J M. XL-1Blue: A high efficiency plasmid transforming recA Escherichia coli K12 strain with beta-galactosidase selection. Biotechniques 1987;5:376-8), following the conditions described by Sambrook et al. (Sambrook J, Fritsch E F, Maniatis T. Molecular cloning: A laboratory manual. New York, USA: Cold Spring Harbor Laboratory Press; 1989), and the plasmids present in the resulting colonies growing on selective medium were screened using restriction analysis. The sequence of several recombinant plasmids from each transformation was verified by automatic Sanger sequencing, and for each reaction a representative molecule was chosen whose sequence matched the expected sequence. These plasmids were denominated pET-scFv 4G2 LH (FIG. 5, Sequence No. 18) for the expression of the single-chain antibody fragment, pET-TB4G2 LH (FIG. 6A, Sequence No. 19) for the expression of the multimeric sequence, and pET-MA4G2 LH (FIG. 6B, Sequence No. 20) for the expression of the single chain miniantibody carrying the variable regions from antibody 4G2.

These plasmids can be used for the expression in Escherichia coli, through induction with isopropylthiogalactoside (IPTG) and under the T7 promoter, of the proteins coded by the aforementioned synthetic bands (Sequence No. 15, Sequence No. 16 and Sequence No. 17), which, in their respective immature, unprocessed forms (Sequence No. 21, Sequence No. 22 and Sequence No. 23) contain the following elements in an N- to C-terminal direction: For the unprocessed protein scFv 4G2 LH, a) The pelB signal peptide (Sequence No. 24), b) The aminoacids M (Methionine) and A (Alanine), introduced due to the nature of the Nco I site, c) the variable region of the light chain of monoclonal antibody 4G2 (Sequence No. 25), d) a flexible spacer (linker) (Sequence No. 26), e) the variable region of the heavy chain of the monoclonal antibody 4G2 (Sequence No. 27), f) the aminoacids L (Leucine) and E (Glutamic acid), introduced due to the cloning strategy, and g) a C-terminal segment of 6 histidines; for the unprocessed protein TB4G2 LH: a) The pelB signal peptide (Sequence No. 24), b) The aminoacids M (Methionine) and A (Alanine), introduced due to the nature of the Nco I site, c) the variable region of the light chain of monoclonal antibody 4G2 (Sequence No. 25, d) d) a flexible spacer (linker) (Sequence No. 26), e) the variable region of the heavy chain of the monoclonal antibody 4G2 (Sequence No. 27), f) a flexible spacer (linker) (Sequence No. 28), g) a fragment from human matrilin that confers the molecule the property of being able to trimerize in solution (Sequence No. 51), h) the aminoacids L (Leucine) and E (Glutamic acid), introduced due to the cloning strategy, and e) a C-terminal segment of 6 histidines; and for the unprocessed MA4G2 LH protein: a) The pelB signal peptide (Sequence No. 24), b) The aminoacids M (Methionine) and A (Alanine), introduced due to the nature of the Nco I site, c) the variable region of the light chain of monoclonal antibody 4G2 (Sequence No. 25), d) a flexible spacer (linker) (Sequence No. 26), e) the variable region of the heavy chain of the monoclonal antibody 4G2 (Sequence No. 27), f) a flexible spacer (linker) composed of three successive glycines (G), g) a fragment of the constant region of the IgG1 human immunoglobulins that contains the hinge and the CH2 and CH3 domains, where the aminoacid C (Cysteine) of the hinge has been changed by mutagenesis to an S (Serine) and the potential glycosylation site of the CH2 domain has been eliminated by mutating an N (Asparagine) to a Q (Glutamine) (Sequence No. 52), h) h) the aminoacids L (Leucine) and E (Glutamic acid), introduced due to the cloning strategy, and e) a C-terminal segment of 6 histidines.

These elements allow the processing of these proteins (scFv 4G2, TB4G2 and MA4G2) through leader peptide cleavage and their secretion to the E. coli periplasm, where the prevalent oxidizing conditions allow their correct folding and formation of their disulphide bridge, and also facilitate their purification using immobilized metal affinity chromatography (IMAC) (Sulkowski, E. (1985) Purification of proteins by IMAC. Trends Biotechnol. 3, 1-7). The final sequences of scFv 4G2, TB4G2 and MA4G2 after posttranslational processing and secretion are shown in Sequence No. 53, Sequence No. 54 and Sequence No. 55.

### Example No. 11

### Expression and Purification of scFv 4G2, TB4G2 and MA4G2

The purification of scFv 4G2, TB4G2 and MA4G2 from plasmids pET-scFv4G2 LH, pET-TB4G2 LH y pET-MA4G2, respectively, used the process described as follows: The corresponding plasmid was transformed following the instructions of Sambrook et al. (Sambrook J, Fritsch E F, Maniatis T. Molecular cloning: A laboratory manual. New York, USA: Cold Spring Harbor Laboratory Press; 1989) into the BL21(DE3) E. coli strain (Studier, F. W. and B. A. Moffatt. “Use of bacteriophage T7 RNA polymerase to direct selective high-level expression of cloned genes.” J.Mol.Biol. 189.1 (1986): 113-30), and an isolated colony was used to inoculate a 50 mL culture of Luria-Bertani medium supplemented with ampicillin at 50 μg/mL (LBA), which was grown for 12 hours at 30° C. at 350 r.p.m. With this culture, 1 L of LBA medium was inoculated to a starting optical density at 620 nm (OD620) of 0.05, which was then grown for 8 h at 28° C. until late exponential phase. This culture was then induced by the addition of isopropylthiogalactoside (IPTG), and grown in the same conditions for an additional period of 5 hours.

The culture obtained as described above was centrifuged at 5000×g for 30 min. at 4° C. and the periplasmic fraction was extracted from the resulting biomass using the method of Ausubel et al. (Ausubel, F. M., Brent, R., Kingston, R. E., Moore, D. D., Seidman, J. G., Smith, J. A. and Struhl, K (1989) in Current Protocols in Molecular Biology. John Wiley & Sons, New York). This fraction was dialyzed against 50 mM phosphate buffer pH 7/20 mM imidazole using a membrane with a 1000 Da cut-off, and protein PMEC1-His6 was obtained from the dialyzate by immobilized metal affinity chromatography (Sulkowski, E. 1985, Purification of proteins by IMAC. Trends Biotechnol. 3, 1-7), using Ni-NTA agarose (Qiagen Benelux B. V., The Netherlands) following the instructions of the manufacturer

### Example 12

### Neutralization of Viral Infection by MAG4G2 and TB4G2 Proteins

The characterization of the biological activity of MA4G2 y TB4G2 chimeric proteins was performed using a plaque reduction neutralization assay in BHK-21 cells (Table 10). This same assay was employed to compare the biological activity of chimeric proteins with Mab 4G2, its Fab and Fab2 fragments and scFv4G2 (Table 10).

Fab and Fab2 fragments were obtained by digestion with papain and pepsin of Mab 4G2. After protease digestion Fab and Fab2 were separated from the Fc fragment by affinity chromatography with immobilized protein A. Fab and Fab2 isoforms were further purified by ion exchange chromatography. Neutralizing titers were defined as the dilution of the molecule yielding 50% reduction of the viral plaque number. Dilution of the different molecules was adjusted to obtain equimolar concentration in the assays.

TABLE 10
Viral neutralization assay of MA4G2, TB4G2, Mab4G2
and Mab4G2 Fab, Fab2 and scFv4G2 fragments.

| | Viral neutralization titer* | | | |
|---|---|---|---|---|
| | anti- | anti- | anti- | anti- |
| Molecule | DEN-1 | DEN-2 | DEN-3 | DEN-4 |
| Mab 4G2 | 1:1280 | 1:1280 | 1:320 | 1:128 |
| Fab 4G2 | 1:1280 | 1:1280 | 1:320 | 1:128 |
| Fab2 4G2 | 1:1280 | 1:1280 | 1:320 | 1:128 |
| scFv4G2 | 1:1280 | 1:1280 | 1:320 | 1:128 |
| TB4G2 | 1:128000 | 1:128000 | 1:64000 | 1:32000 |
| MA4G2 | 1:128000 | 1:128000 | 1:64000 | 1:32000 |
| Mab Hep1 | <1:40 | <1:40 | <1:40 | <1:40 |

*Neutralizing titers were defined as the dilution yielding 50% reduction of viral plaques in BHK-21 cells.

1. A topographic and highly conserved area, characterized by being exposed on the mature virion, and which represents an epitope shared among flaviviruses, that can be used in the development of wide-spectrum molecules for the prevention and/or treatment of infections due to Dengue Virus 1-4 and other flaviviruses.

2. A topographic and highly conserved area according to claim 1, wherein this area is an epitope of protein E from the envelope of flaviviruses that is defined by the following residues of the E protein from Dengue Virus 2 or the corresponding epitopes from other flaviviruses: ASN67, THR69, THR70, SER72, ARG73, CYS74, LEU82, GLU84, GLU85, ASP87, VAL97, ARG99, GLY100, TRP101, GLY102, ASN103, GLY104, CYS105, GLY106, MET118, HIS244, LYS246, LYS247, GLN248, VAL252.

3. A topographic area according to claim 1, characterized by being exposed on the surface of the E protein of the following flaviviruses: West Nile Virus, St. Louis Encephalitis Virus, Dengue1, Dengue2, Dengue3, Dengue4, Japanese Encephalitis Virus, Kunjin Virus, Kyasanur Forest Disease Virus, Tick-borne Encephalitis Virus, Murray Valley Virus, LANGAT virus, Louping III Virus and Powassan virus.

4. Molecules according to claim 1, useful for the prevention and/or treatment of the infections due to Dengue Virus 1-4 and other flaviviruses, based on the topographic area described in claim 1, characterized by their ability to induce a response of neutralizing antibodies which cross-react with the four serotypes of Dengue Virus and other flaviviruses in individuals immunized with said molecules.

5. Molecules according to claim 4, wherein said molecules are recombinant proteins or chimeric peptides with Sequence identification numbers 14 and 29-50.

6. Protein molecules according to claim 4, whose primary structure is defined by the sequence A-B-L-C wherein A is the sequence of a peptide of 0 to 30 aminoacids, B is the sequence of the fragment Leu237-Val252 of protein E from Dengue Virus 2 or the homologous sequence from Dengue Virus 1, 3, 4, or any other flavivirus, L is a sequence of 3 to 10 aminoacids that functions as a stabilizing linker, and C is the sequence of the fragment Lys64-Thr120 of protein E from Dengue Virus 2 or the homologous sequence from Dengue Virus 1, 3, 4, or any other flavivirus.

7. Protein molecules according to claim 4, whose primary structure is defined by the sequence A-C-L-B wherein A is the sequence of a peptide of 0 to 30 aminoacids, B is the sequence of the fragment Leu237-Val252 of protein E from Dengue Virus 2 or the homologous sequence from Dengue Virus 1, 3, 4, or any other flavivirus, L is a sequence of 3 to 10 aminoacids that functions as a stabilizing linker, and C is the sequence of the fragment Lys64-Thr120 of protein E from Dengue Virus 2 or the homologous sequence from Dengue Virus 1, 3, 4, or any other flavivirus.

8. A protein according to claim 6 wherein A is a bacterial signal peptide.

9. A protein according to claim 6 wherein A is a yeast or mammalian signal peptide.

10. A synthetic or recombinant fusion protein, characterized by being formed by the molecule described in claim 4, and an N- or C-terminal fusion to one or more peptide or protein fragments that enhance its protective or therapeutic effect and/or facilitate its purification and/or detection.

11. A recombinant or synthetic fusion protein according to claim 10, wherein the N- or C-terminal fusion partner is one or more peptide or protein fragments containing helper T-cell epitopes.

12. A recombinant or synthetic fusion protein according to claim 10, wherein the N- or C-terminal fusion partner is a Histidine tag.

13. A nucleic acid coding for a protein corresponding to claim 4.

14. A prokaryote or eukaryote host cell containing a nucleic acid according to claim 13.

15. A pharmaceutical composition characterized by containing one or more proteins according to claim 4, capable of inducing on the receiving organism an immune response of neutralizing and protective antibodies which are cross-reactive with Dengue Virus 1-4.

16. A pharmaceutical composition characterized by containing one or more proteins according to claim 4, capable of inducing on the receiving organism an immune response of neutralizing and protective antibodies which are cross-reactive with other flaviviruses.

17. A pharmaceutical composition characterized by being able to induce on the receiving organism an immune response of neutralizing and protective antibodies which are cross-reactive with Dengue Virus 1-4 and other flaviviruses, based on the use of live vectors or naked DNA containing genes coding for the proteins described in claim 4.

18. Synthetic or recombinant protein or peptide molecules according to claim 5, useful as diagnostic reagents for the detection of anti-flavivirus antibodies.

19. Molecules according to claim 1, useful for the prevention and/or treatment of infections due to Dengue Virus 1-4 and other flaviviruses, based on the topographic area described in claim 1, characterized by their ability to prevent or attenuate the viral infection due to their interaction with said topographic area.

20. Molecule according to claim 19, wherein said molecule is a human antibody or an antibody produced in other species.

21. An antibody according to claim 20, wherein said antibody is cross-reactive with different flaviviruses and is neutralizing for viral infection.

22. Molecule according to claim 19 to be used in the prevention and/or treatment of infections due to Dengue Virus 1-4 or other flaviviruses, wherein said molecule is a recombinant or proteolytic fragment of the antibody.

23. A molecule according to claim 22, wherein said molecule is a recombinant single-chain Fv fragment of the antibody (scFv).

24. A molecule according to claim 23, characterized by being linked, with or without spacers, to a protein sequence that confers said molecule the ability to assemble as a molecule with polyvalent binding to mature virions.

25. A molecule according to claim 24, wherein the protein sequence linked to said molecule contains a spacer and the hinge, CH2 and CH3 regions of a human immunoglobulin, with the sequence specified in Sequence No. 55 and 56.

26. A molecule according to claim 24, wherein the protein sequence linked to said molecule contains a spacer and a trimerization domain with the sequence specified in Sequence No. 54.

27. A nucleic acid coding for a protein corresponding to claim 18.

28. A prokaryote or eukaryote host cells containing a nucleic acid according to claim 27.

29. A pharmaceutical composition characterized by containing one or more proteins according to claim 18, capable of preventing or attenuating the infection due to Dengue Virus 1-4.

30. A pharmaceutical composition characterized by containing one or more proteins according to claim 18, capable of preventing or attenuating the infection due to other flaviviruses.

31. Synthetic or recombinant protein or peptide molecules according to claim 18, useful as diagnostic reagents for the detection of flaviviruses.

32. A molecule useful as a wide-spectrum therapeutic candidate against flaviviruses that is identified with a method that comprises the contact of said molecule with the area or conserved epitope of protein E according to claim 1, wherein this contact or binding indicates that said molecule is a wide-spectrum therapeutic candidate.

33. A molecule according to claim 32, wherein said molecule is selected among the following classes of compounds: proteins, peptides, peptidomimetics and small molecules

34. A method according to claim 32, wherein said molecule in included in a library of compounds.

35. A method according to claim 34, wherein said library of compounds is generated by combinatorial methods.

36. A method according to claim 32, wherein said contact is measured by an in vitro assay.

37. A method according to claim 36, wherein said assay is performed by blocking the binding of molecules by preventing or attenuating viral infection due to interaction with a conserved area characterized by being exposed on the mature virion, and which represents an epitope shared among flaviviruses, that can be used in the development of wide-spectrum molecules for the prevention and/or treatment of infections due to Dengue Virus 1-4 and other flaviviruses.

38. A method according to claim 36, wherein said method is performed by blocking the binding of molecules by preventing or attenuating viral infection due to interaction with protein molecules characterized by their ability to induce a response of neutralizing antibodies which cross-react with the four serotypes of Dengue Virus and other flaviviruses in individuals immunized with said molecules.

39. A method according to claim 32, wherein said union is measured by an in vivo assay.

40. A method according to claim 32, wherein said method is a computer-aided method that comprises: 1) the atomic coordinates corresponding to the residues that form the highly conserved area of protein E characterized by being exposed on the mature virion, and which represents an epitope shared among flaviviruses, that can be used in the development of wide-spectrum molecules for the prevention and/or treatment of infections due to Dengue Virus 1-4 and other flaviviruses, and said coordinates are available on protein structure databases or modeled by computational means or experimentally determined, 2) the atomic coordinates of molecules, which have been determined experimentally or modeled by computational means, 3) a computational procedure of molecular docking that allows the determination of whether this molecule will be able to make contact with the highly conserved area characterized by being exposed on the mature virion, and which represents an epitope shared among flaviviruses, that can be used in the development of wide-spectrum molecules for the prevention and/or treatment of infections due to Dengue Virus 1-4 and other flaviviruses.
